(19) United States
(12) Patent Application Publication
(10) Pub. No.: US 2016/0235574 A1
(43) Pub. Date: Aug. 18, 2016

(54) LUMBAR SUPPORT DEVICE

(71) Applicant: Victor M. HAYES, Odessa, FL (US)

(72) Inventor: Victor M. HAYES, Odessa, FL (US)

(21) Appl. No.: 14/993,241

(22) Filed: Jan. 12, 2016

Related U.S. Application Data
(60) Provisional application No. 62/102,568, filed on Jan. 12, 2015.

(51) Int. Cl. A61F 5/02; A61B 5/00; A61F 5/30

(52) U.S. Cl. CPC A61F 5/028; A61F 5/30; A61B 5/0011; A61B 5/0004

(57) ABSTRACT

Provided are adjustable, modular lumbar support devices that may be used to provide lumbar and/or belly support for mammals having belly weight. According to non-limiting example embodiments, support or brace devices provided herein may be worn by pregnant women at various stages throughout a woman's pregnancy, and/or post-partum and may be adjusted to naturally contour/conform to the woman's anatomy, which changes throughout pregnancy. Also provided herein are methods of preventing and/or reducing back pain in a mammal that include applying the lumbar support to a mammal as directed. Further provided are kits that include at least one lumbar support device provided herein, which kit may optionally include instructions for proper application and/or adjustment of the lumbar support device.

### RELATED APPLICATION

This application claims the benefit of U.S. Provisional Application No. 62/102,568, filed on Jan. 12, 2015, which is hereby incorporated by reference herein in its entirety.

### TECHNICAL FIELD

Present embodiments relate generally to lumbar support devices and methods of treating and/or preventing back pain in a person, due to for example pregnancy or other belly weight, and/or reduced core strength in the person. Further provided are methods of using such devices, and kits including such lumbar support devices.

### BACKGROUND ART

Back pain is a common complaint during pregnancy. Typically a woman's body will gain between 15-40 pounds. Pregnancy causes the body's hormones to fluctuate causing many physiologic changes throughout the body. The key hormones that dramatically fluctuate during pregnancy are estrogen, progesterone and relaxin. Fluctuations in both estrogen and progesterone are necessary for a woman to become pregnant and for an expecting mother to carry her baby to term. During pregnancy high progesterone levels function to maintain a healthy internal environment for the baby. However, as the progesterone and relaxin levels increase during pregnancy, joints, ligaments, tendons and tissues throughout the body relax and stretch.

Once the joints and ligaments relax, the lower back becomes more flexible and the body naturally changes the spinal alignment to compensate for the excess “belly weight” throughout pregnancy. However, the added “belly weight” is like carrying a heavy object in front of the lower pelvis.

In objects with an irregular or changing shape, as in the human body during pregnancy, the center of gravity changes throughout different stages of pregnancy. Typically in a normal, healthy, standing individual, the center of gravity is located about 1 cm behind the junction of the lumbar spine and sacrum. However, during pregnancy, the excess “belly weight” shifts the center of gravity forward several inches.

This added weight during pregnancy changes the center of gravity, so one's body compensates by increasing their lower back's natural curvature called Lordosis. The body's center of gravity is a single point at which gravity exerts its downward force. For one to balance and stand without falling over, a person's center of gravity must be balanced by coordinating and using their different muscle groups (especially core muscles). Lordosis is a backward curvature of spine and helps to counter balance the change in the center of gravity.

During pregnancy the combination of the increased “belly” weight, increase in spinal flexibility, change in the center of gravity, and altered lumbar lordosis, create a delicate equilibrium. Any additional activities or postural changes that create undo stress on the lumbar spine can lead to imbalance in the equilibrium. Ultimately all of these changes and this imbalance cause muscle fatigue. This muscle fatigue is a common cause of pregnancy related back pain and can develop into a more serious spinal disorder if untreated. Even in fit women lower back pain is a painful reality that will affect 50-80 percent of all pregnant women.

Another result of pregnancy and giving birth is that incisions (in the case of a C-section) or stretching of lower abdominal muscles (in a vaginal delivery), cause weakened core muscles, which also lead to back pain post-partum.

Typical treatments for back pain include administration of anti-inflammatories and other medications/treatments, however such medications must be avoided during pregnancy to avoid any harmful affect to the baby. Non-pregnant persons with back pain may also not be able to, or may not choose to, use anti-inflammatories and/or other medications or available treatments.

### SUMMARY

Provided herein are adjustable, modular, lumbar support devices that may be used to provide lumbar support. The present support devices are effective at decreasing the stresses on the spine, and improving posture/alignment during activities of daily living.

According to non-limiting example embodiments, support or brace devices provided herein may be used as a maternity brace throughout a woman's pregnancy (even as the woman's anatomy changes throughout the pregnancy). When used as a maternity brace, the present devices may be used both during pregnancy for gestational support and/or post-partum (post-natal support), because they are adjustable and modular and have safety mechanisms therein to prevent over-tensioning. The present devices may also be used as lumbar support braces for persons who have excess belly weight or persons with ascites, etc.

The brace is modular at least in that it incorporates removable padded, but rigid, anterior and posterior panels.

Also provided herein are methods of preventing and/or reducing back pain in a person that include the person applying a lumbar support device provided herein to their back and securing the device around their belly.

Further provided are kits that include at least one lumbar support device provided herein, which kits may optionally include instructions for proper application and/or adjustment of the lumbar support device. Kits may also include e.g., one or more removable padded panels, e.g. for post-partum or other non-pregnancy uses.

Further provided are systems that include at least one lumbar support device, which includes electronics incorporated therein, which are capable of detecting and/or monitoring one or more maternal and/or fetal vital signs. The electronics may have a method of recording, displaying, and/or communicating such vital signs (or data).

The foregoing has outlined rather broadly the features and technical advantages of the present invention so that the detailed description of the invention that follows may be better understood. Additional features and advantages of the invention will be described herein. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. herein is provided for the purpose of illustration and description only and is by no means intended to define the limits the invention.

### BRIEF DESCRIPTION OF THE DRAWINGS

Non-limiting example embodiments described herein, with reference to the following accompanying Figures.

FIGS. 1A and 1B depict front/anterior perspective views of an example lumbar support device 20 provided herein. FIG. 1C is a front/anterior view of the lumbar support device 20. FIG. 1D is a side view of the lumbar support device of FIGS. 1A-1C. FIG. 1E is a top view of the lumbar support device of FIGS. 1A-1C. FIG. 1F depicts a perspective view of the example lumbar device.

FIG. 2 depicts an exploded view of components of a non-limiting example lumbar support device 20 of FIGS. 1A-1E.

FIGS. 3A, 3B, and 3C depict a layer 1 of fabric from posterior view (FIG. 3A) of the lumbar support device of FIGS. 1A-2. FIG. 3B depicts a side view of layer 1. FIG. 3C depicts a perspective view of the example layer 1.

FIGS. 4A, 4B, and 4C depict a second layer (from the exploded view of FIG. 2) of the lumbar device of FIGS. 1A-2. In particular, FIG. 4A depicts a padding layer 2. FIG. 4B depicts a cross section of the padding layer 2. FIG. 4C depicts a perspective view of the example padding 2.

FIGS. 5A, 5B, and 5C depict one of several wings 4 (e.g. mesh wings from front and side perspectives, respectively), which are next in the exploded view (see FIG. 2) of the lumbar support device of FIGS. 1A-2. FIG. 5C depicts a perspective view of the example wing 4.

FIGS. 6A, 6B, and 6C depict a wing 4 of FIG. 5A with multiple ribs 5 thereon from both a front and a side view (FIGS. 6A and 6B respectively). The depicted wing 4 with ribs 5 prior to adding another wing 4 thereon. FIG. 6C depicts a perspective view of the example wing ribs 5.

FIGS. 7A, 7B, 7C, and 7D depict an example corset pulley system assembly according to non-limiting embodiments of the lumbar device of FIGS. 1A-2. In particular, FIG. 7A depicts an example of the corset pulley system assembly which will be enclosed within the material of the brace (from a front view), FIG. 7B from a top view, and FIG. 7C from a right side view. FIG. 7D depicts a perspective view of the example corset sub-assembly 6.

FIG. 8A further depicts the corset pulley system assembly as shown in FIGS. 7A-7C and other FIGS. herein in an exploded view. FIG. 8B depicts a perspective view of the example corset assembly.

FIGS. 9A and 9B depict a front and a side view of a corset fabric 23 of a corset assembly according to inter alia, FIG. 8A. FIG. 9C depicts a perspective view of the example corset fabric 23.

FIGS. 10A and 10B depict a front and a side view of a corset fabric 24 of a corset assembly according to inter alia, FIG. 8A. FIG. 10C depicts a perspective view of the example corset fabric 24.

FIGS. 11A and 11B depict a front and a side view of one of the one of multiple diagonal corset fabric portions of a corset assembly according to inter alia, FIG. 8A. FIG. 11C depicts a perspective view of the example corset fabric 22.

FIGS. 12A and 12B depict the corset assembly, and in particular, the stitching of fabrics together along the hatched area. FIG. 12C depicts a perspective view of the example right side assembly of the corset fabric construction.

FIGS. 13A and 13B depict the corset assembly, and in particular, the stitching of fabrics together along the hatched area. FIG. 13C depicts a perspective view of the example left side assembly of the corset fabric construction.

FIGS. 14A and 14B depict a front and a side view of a corset bone of a corset assembly according to inter alia, FIG. 8A. FIG. 14C depicts a perspective view of the example corset bone 25.

FIGS. 15A, 15B and 15C depict a front, top and side view, respectively of a clip of a corset assembly according to inter alia, FIG. 8A. FIG. 15D depicts a perspective view of the example clip 21.

FIG. 16A depicts an exploded front perspective view of a wing assembly according to non-limiting embodiments of the lumbar device of FIGS. 1A and 2. FIG. 16B depicts a perspective view of the example wing assembly. Although labeled “blue wing”, it should be noted that none of the colors indicated herein are meant to be limiting or binding and further example embodiments include other colors.

FIGS. 17A and 17B depict a front view and view from the side respectively, of one of the wings 8 from e.g. FIG. 16A. FIG. 17C depicts a perspective view of the example wing.

FIGS. 18A, 18B and 18C depict a piece of center fabric 10, from front, top and side views, respectively, arranged between the wings 8 as shown in FIG. 16A. FIG. 18D depicts a perspective view of the example center fabric 10.

FIGS. 19A, 19B and 19C depict a slit bone 7 from FIG. 16A, arranged from front, top and side views, respectively. FIG. 19D depicts a perspective view of the example slit bone 7.

FIG. 20 depicts an exploded view of an example embodiment of one or two arms having layers 11-17 and an attachment 18 to enable attachment of the arm to a second arm in accordance with the device of FIGS. 1A and 2 et seq.

FIGS. 21A, 21B and 21C depict wing fastener portions 3 (e.g. Velcro®), which may attach one wing to the other wing to secure the wings around the belly in a desired location. FIG. 21D depicts a perspective view of the example wing Velcro®.

FIGS. 22A, 22B and 22C depict front, top and side views of arms 17 from the device of FIGS. 1A-2. FIG. 22D depicts a perspective view of the example arm template. Embodiments of FIG. 22A-22D may have the following components.

| ITEM NO. | PART NAME | MATERIAL | QTY. |
|---|---|---|---|
| 11 | BLACK COTTON ARM | COTTON | 2 |
| 12 | ARM PADDING | COTTON | 2 |
| 13 | BLACK MESH ARM | MESH | 4 |
| 17 | BLUE ARM | POLAR FLEECE | 2 |

FIGS. 23A, 23B and 23C depict front, top and side view of an elastic arm strap 16, which is a component of each arm, as shown in FIG. 20. FIG. 23D depicts a perspective view of the example arm strap.

FIG. 24A depicts an example wing 4 (titled “black mesh wing”, but not limited to black or mesh) configured for assembly. In particular, it is configured such that the arm strap 16 (not shown) must be pulled through the transparent “blue square” (not necessarily limited to this or any color) prior to sewing it to the hatched area on the diagram. The “blue” square and slit 19 is transparent and located in the center of the wing 4 for reference. This connects the arm strap when one is building the brace). FIG. 24A details embodiments of how to assemble the arm when fabricating the brace, i.e., that it is necessary to pull the arm through the blue square slit hole prior to sewing it, because once it is sewed in, it will be impossible to get the arm through. FIG. 24B depicts a perspective view of the example arm strap with blue wing assembly. The end of the constructed arm strap may be stitched. Make sure that it is passed through the outer hole of the clip before sewing. Embodiments of FIG. 24A may have the following components.

| ITEM NO. | PART NAME | MATERIAL | QTY. |
|---|---|---|---|
| 4 | BLACK MESH WING | MESH | 4 |
| 19 | BLUE SQUARE | POLAR FLEECE | 2 |
| 16 | ARM STRAP | ELASTIC BAND | 2 |

FIGS. 25A, 25B and 25C depict front, top and side views of an arm bone component of the arms according to non-limiting example embodiments of the present invention as shown in FIG. 20. FIG. 25D depicts a perspective view of the example arm bone 14.

FIGS. 26A, 26B and 26C depict front, top and side views of a second arm bone of the arms of the present invention. FIG. 26D depicts a perspective view of the example arm bone 15.

FIGS. 27A, 27B, and 27C depict front, top and side views of black mesh arm 13, with relative placement of arm bones 14 and 15 shown. FIG. 27D depicts a perspective view of the example arm bone placement in the mesh arm.

FIGS. 28A, 28B and 28C depict arm fastener portions 18 (e.g. Velcro®), which may attach one arm to the other arm to secure the arms around the belly. FIG. 28D depicts a perspective view of the example arm Velcro®.

FIGS. 29-34 depict, removable padded, but rigid, anterior and posterior panels, which may be added or removed from the device of FIG. 1 et seq. by the user; e.g., during post-partum or other non-pregnancy use.

FIG. 29 depicts a front view of a removable padded, but rigid, posterior panel in accordance with non-limiting example embodiments.

FIG. 30 depicts a back view of the removable padded posterior panel of FIG. 29.

FIG. 31 depicts a front view of a removable padded, but rigid, coronal side panel in accordance with non-limiting example embodiments.

FIG. 32 depicts a back view of the removable coronal side panel of FIG. 31.

FIG. 33 depicts a front view of a removable padded, but rigid, anterior panel in accordance with non-limiting example embodiments.

FIG. 34 depicts a back view of the removable anterior panel of FIG. 33.

FIGS. 35-40 depict example embodiments of a device of FIG. 1 et seq., and depict the device in use by various users, in accordance with non-limiting examples.

FIG. 35 depicts a close up view of a portion of a lumbar support device in accordance with non-limiting example embodiments. In particular, depicted is an arm emerging from a slit in a wing of the support device.

FIG. 36 depicts one side of a lumbar support device in accordance with non-limiting example embodiments of the present invention. As shown in FIG. 36, an arm emerges from a slit in the corresponding wing.

FIG. 37 depicts a middle portion of an underside of the lumbar support device in accordance with non-limiting example embodiments.

FIG. 38 depicts a device in accordance with the present invention in use as applied to a pregnant user. In the depicted embodiment, the wings are first applied over the belly and attached to one another with sing fasteners, and the arms are applied and positioned over the wings and attached to one another with arm fasteners.

FIG. 39 depicts a device in accordance with the present invention in use as applied to a different pregnant user than in FIG. 38. In this embodiment, the wings are applied toward the bottom of the belly and attached to one another and the arms are positioned over the top of the belly and attached to each other with arm fasteners in that position.

FIG. 40 depicts a device in accordance with the present invention in use as applied to a pregnant user. In the depicted embodiment, the wings are first applied over the belly and attached to one another, and the arms are applied and positioned at least somewhat over the wings and attached to one another over the wings with arm fasteners.

FIG. 41 depicts the front of a posterior panel in accordance with non-limiting example embodiments.

FIG. 42 depicts an anterior panel attached to an arm of the present device, in accordance with non-limiting example embodiments.

FIG. 43 depicts example lumbar support devices of the present invention, of various sizes, in accordance with non-limiting example embodiments.

FIG. 44 depicts an anterior panel in accordance with non-limiting example embodiments.

FIG. 45 depicts an opposite side of an anterior panel of FIG. 44, in accordance with non-limiting example embodiments. The rectangular portion in the middle is a portion of VELCRO®. According to other example embodiments, the device may include a loop fastener or other suitable fastener.

FIG. 46A and 46B depict the front and back of a posterior panel in accordance with non-limiting example embodiments of the invention.

FIG. 47 depicts a non-limiting example of a support device/brace in accordance with the present invention, having one or more coronal panels. In particular, the figure depicts a support device/brace in use on an individual, which has coronal panels inserted (slid in) on both sides.

FIGS. 48A and 48B depict non-limiting examples of coronal panels that may be used in conjunction with example support devices of the present invention.

### DETAILED DESCRIPTION

Generally provided herein are support devices that may be useful for prevention and/or treatment of back pain in a person. By way of non-limiting example, the present embodiments may include devices for prevention of back pain in pregnant female humans or other persons that may for example, have excess belly weight, or persons that may have for example, Ascites.

Also provided herein are kits that include the present devices, and methods of preventing and/or reducing back pain in a person that include applying the present devices to a person in need thereof. A person in need of the present devices may include for example, pregnant female humans, persons having ascites, and/or persons having excess weight or bulk around the belly area.

Additional aspects, advantages and/or other features of example embodiments will become apparent in view of the following detailed description, taken in conjunction with the accompanying drawings. It should be apparent to those skilled in the art that the described embodiments provided herein are merely exemplary and illustrative and are not limiting. Numerous embodiments of modifications thereof are contemplated as falling within the scope of this disclosure and equivalents thereto.

Any references mentioned in this specification are indicative of the level of those skilled in the art to which the invention pertains. Any references herein are incorporated by reference to the same extent as if each was specifically and individually indicated as having been incorporated by reference in its entirety.

Unless otherwise noted, technical terms are used according to conventional usage.

As used herein, “a” or “an” may mean one or more. Also as used herein in the claim(s), when used in conjunction with the word “comprising”, the words “a” or “an” may mean one or more than one. As used herein “another” may mean at least a second or more. Furthermore, unless otherwise required by context, singular terms include pluralities and plural terms include the singular.

Provided herein are adjustable, lumbar support devices. Example embodiments include lumbar support devices that may include one or more detachable padded panels to provide additional support. In particular, provided herein are lumbar support devices that include at least two wings having wing fasteners to enable the wings to fasten to each other across a belly of a user; and at least two arms connected to each other by an elastic corset assembly. The elastic corset assembly may be configured to allow adjusting the straps to the appropriate tension and location, while preventing over tensioning. The arms emerge from slits in the wings. The arms have arm fasteners to enable the arms to fasten to each other across, over, or under the belly of the user.

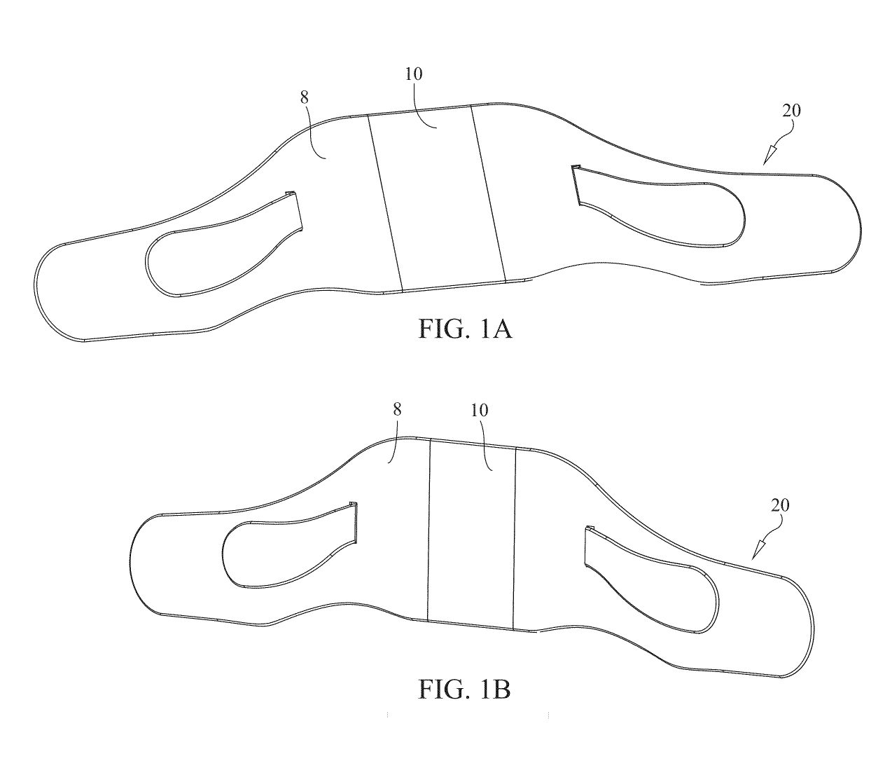

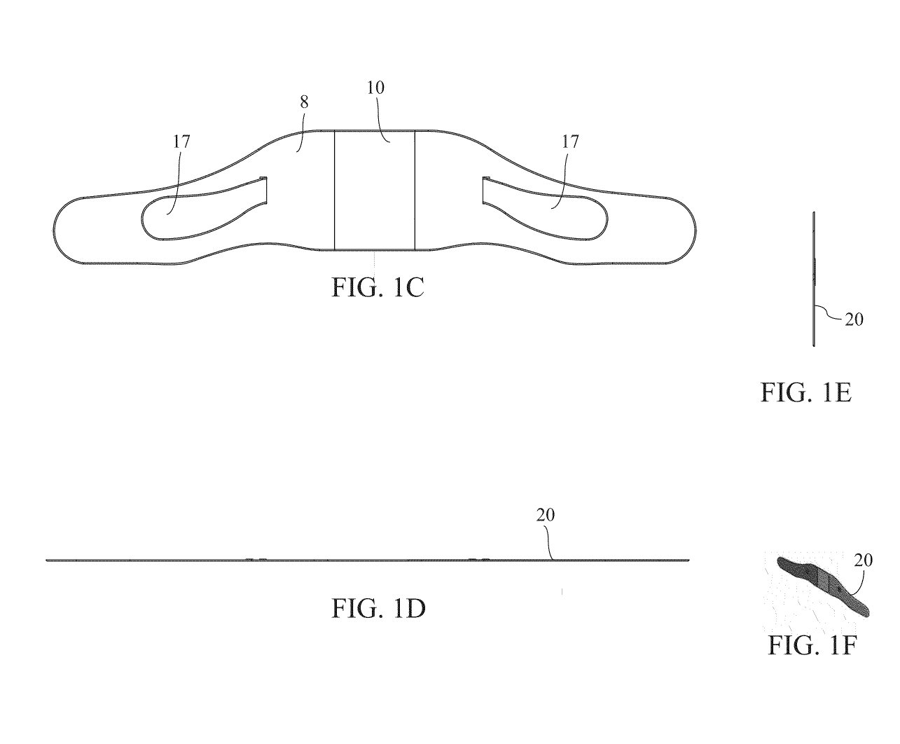

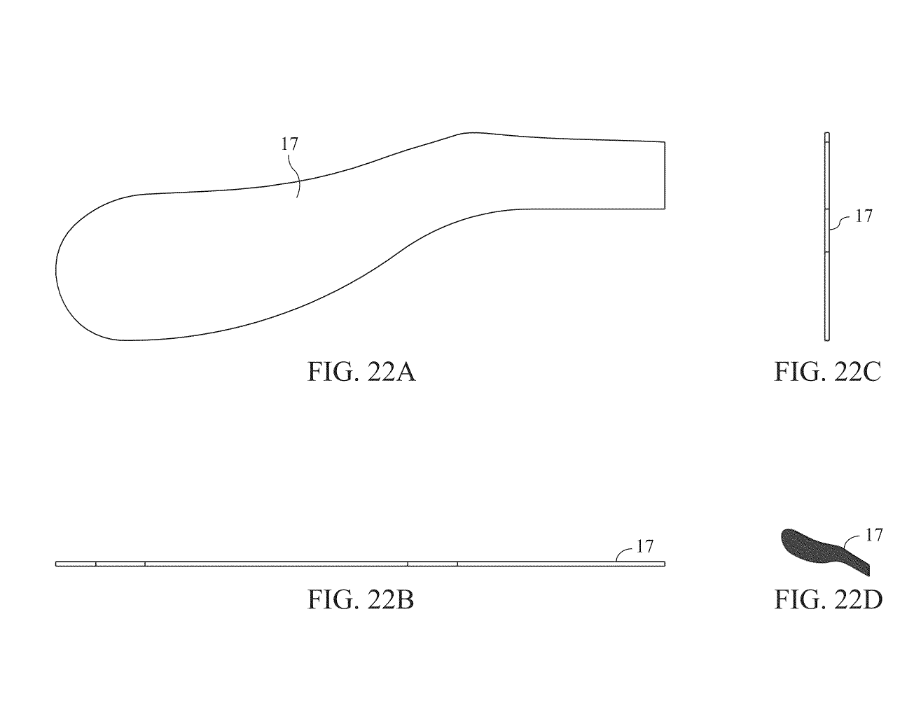

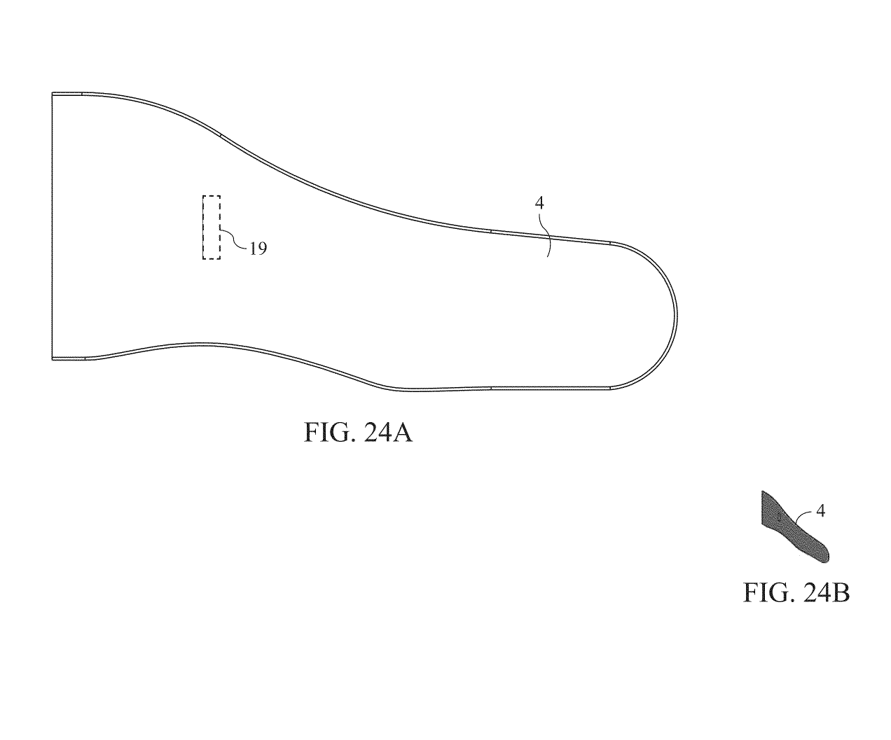

According to non-limiting example embodiments, adjustable lumbar support devices are provided, which may optionally include one or more detachable padded panels for additional support if desired. FIGS. 1A-28C depict various views of non-limiting example embodiments of the present adjustable lumbar supports, without depicting the optional modular padded panels which may be added thereto.

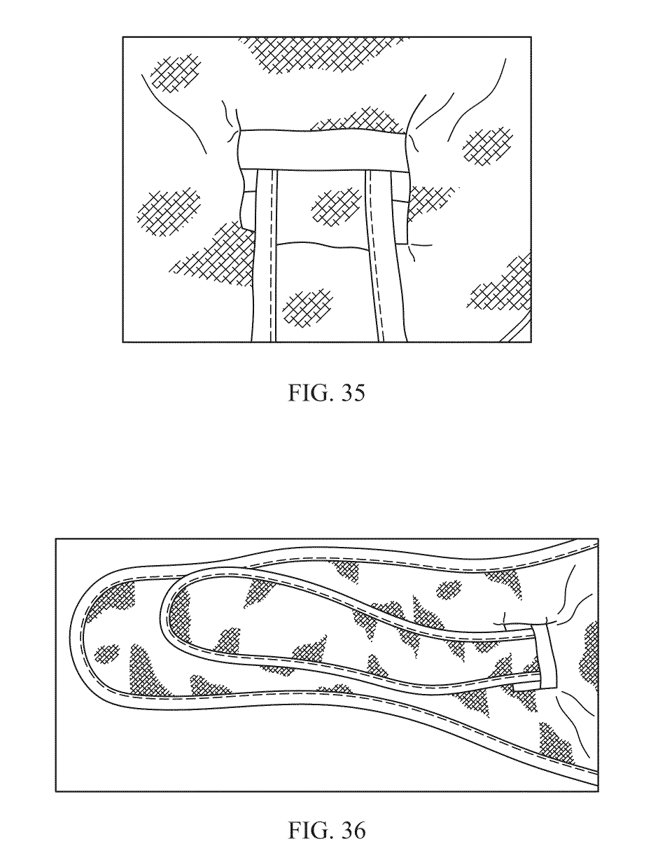

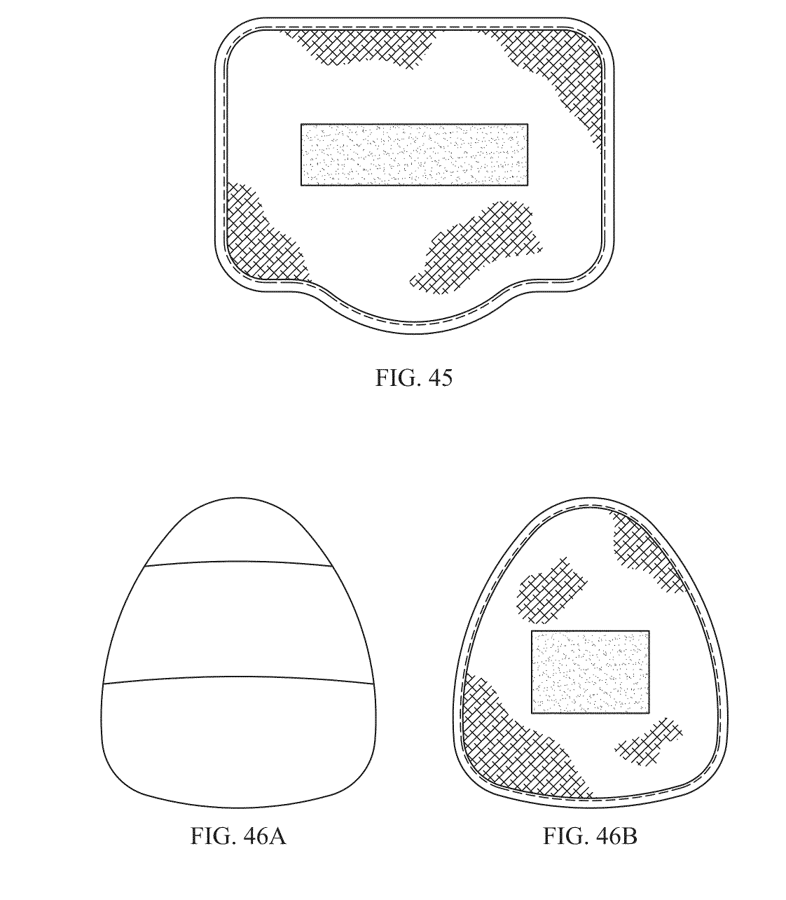

FIGS. 29-34, 41-42, 44-46B, 48A and 48B depict example detachable padded panels. FIGS. 35-40 and 47 are example devices provided herein including figures of the device in use.

FIGS. 1A and 1B depict front/anterior perspective views of an example lumbar support device 20 provided herein. These views depict, inter alia, first and second right and left wings 8 of the device that are connected there-between by one or more material portion 10. The fabric portion 10 may include various materials including for example lycra and cotton.

The depicted device shows first and second arms 17 that protrude from slits in the wings on either side of the device. FIG. 1C is a front/anterior view of the lumbar support device 20. The arms may be pulled around either side of a user and secured around, above or below a user's belly.

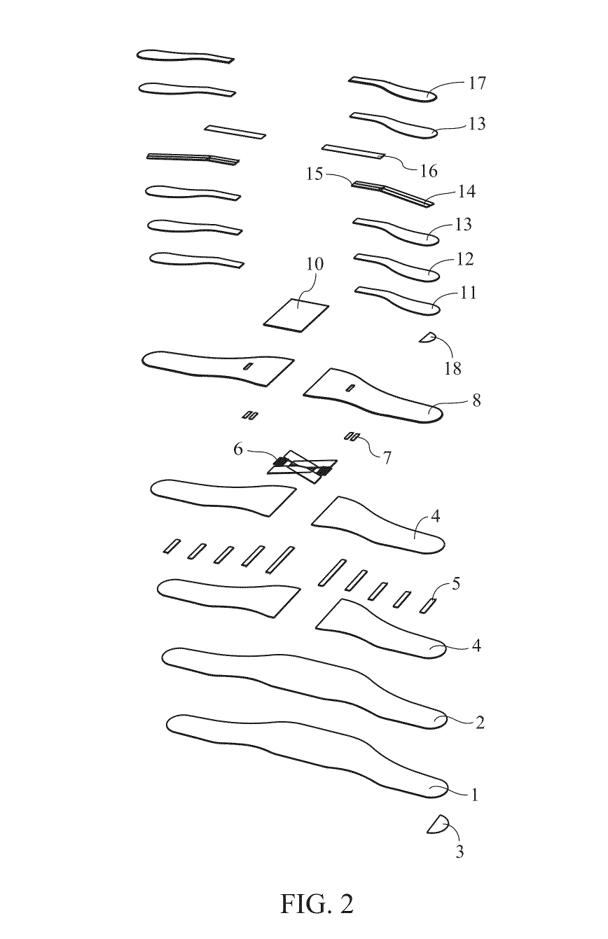

FIG. 2 depicts an exploded view of components of a non-limiting example lumbar support device 20 provided herein. In particular FIG. 2 shows various layers of fabric, mesh, lycra, boning, elastic, and other components of the wings, arms, corset pulley system assembly, etc. of example embodiments.

The depicted embodiments include the following layers: A first cotton layer, which is a base layer of wings of the present device, the bottom side of layer 1 is the portion of the device that contacts a user upon application of the device to a user. Next a padding layer 2 is provided. Next is a wing rib assembly having e.g., four mesh wings 4 with wing ribs 5 there-between. A corset pulley system assembly 6 is provided which attaches to ends of arms (layers 11-17). The arms are fed through slits in wings. The elements of FIG. 2 may be for example, as follows:

| ITEM NO | PART NAME | MATERIAL | QTY. |
|---|---|---|---|
| 1 | BLACK COTTON | COTTON | 1 |
| 2 | PADDING | COTTON | 1 |
| 3 | WING VELCRO | VELCRO | 1 |
| 4 | BLACK MESH WINGS | MESH | 4 |
| 5 | WING RIBS | RIGILENE | 2 |
| 6 | CORSET SUB- | N/A | 1 |
| 7 | SLIT BONE | RIGILENE | 4 |
| 8 | BLUE WINGS | POLAR FLEECE | 2 |
| 10 | CENTER FABRIC | COTTON | 1 |
| 11 | BLACK COTTON ARM | COTTON | 2 |
| 12 | ARM PADDING | COTTON | 2 |
| 13 | BLACK MESH ARM | MESH | 4 |
| 14 | ARM BONE 1 | RIGILENE | 4 |
| 15 | ARM BONE 2 | RIGILENE | 4 |
| 16 | ARM STRAP | ELASTIC BAND | 2 |
| 17 | BLUE ARM | POLAR FLEECE | 2 |
| 18 | ARM VELCRO | VELCRO | 1 |

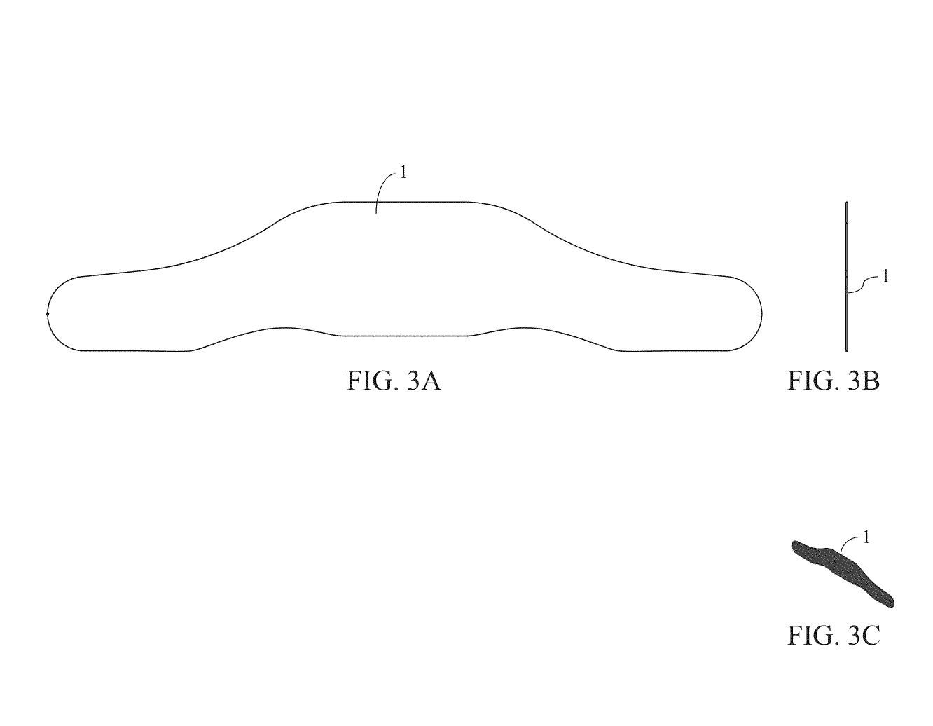

FIGS. 3A and 3B depict layer 1 of fabric (e.g. cotton) from a posterior view (FIG. 3A) of the lumbar support device of FIGS. 1A-2. FIG. 3B depicts a side view of layer 1.

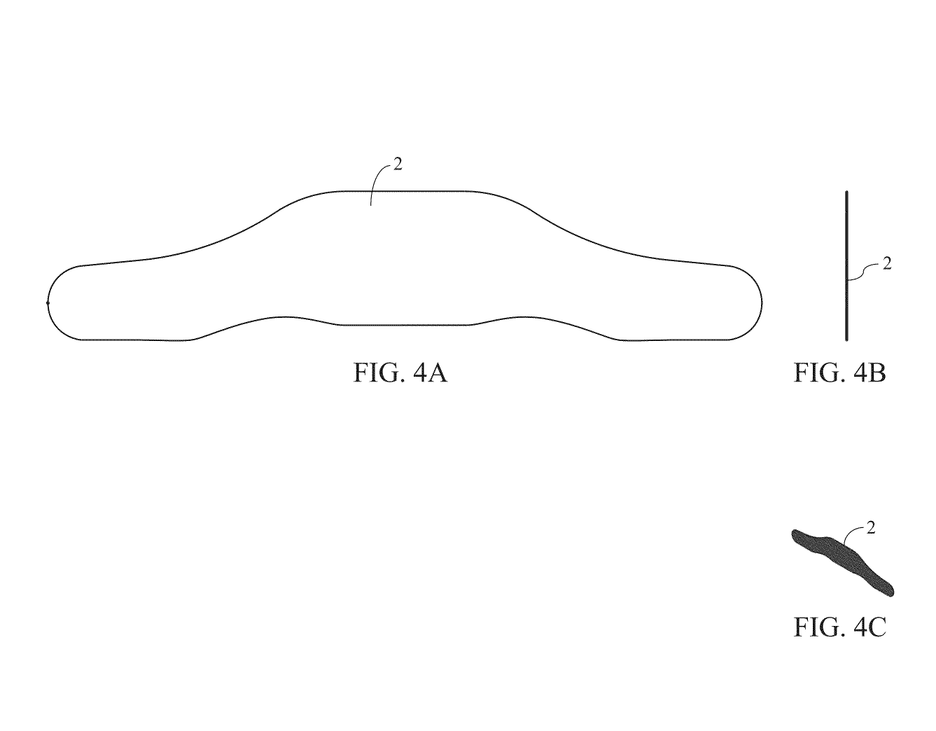

A second padding layer 2 (from the exploded view of FIG. 2) of the lumbar device is depicted in FIG. 4A. The padding layer may be made for example of cotton, and may be formed to essentially be the same size and shape of the fabric layer 1, or may be made to be smaller than such fabric layer 1. FIG. 4B depicts a cross section of the padding layer 2. In alternative embodiments, padding 2 may be eliminated in favor of for example, a thicker layer 1 (if desired). Or according to other embodiments, additional padding layers may be added.

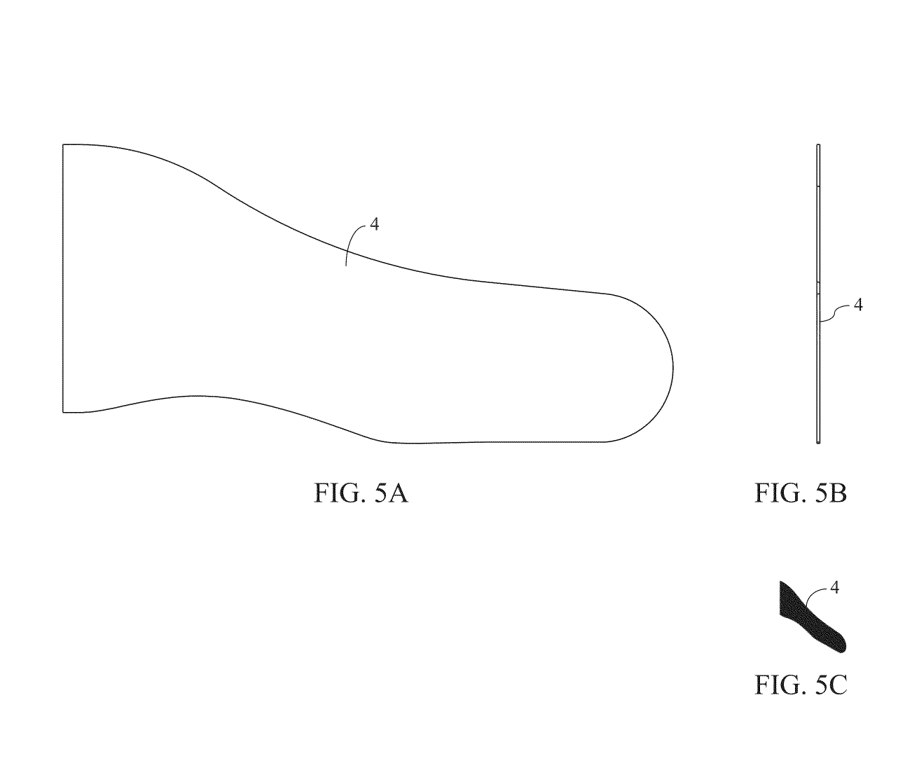

A mesh wing 4 is shown in FIGS. 5A and 5B. The present devices include several mesh wings 4, including e.g., two on each side of the device. The mesh wings 4 may be configured essentially in size and shape to match the padding 2 and/or layer 1 of the padding and wings.

The wings are also configured to receive a rib or boning material between a set of corresponding wings on each of the left and right sides of the device. That is, as shown in the exploded view (FIG. 2), the right side of the device may have a set of layers as follows: wing 4, ribs 5, wing 4, to form a right wing assembly; and the left side of the device may have a set of layers as follows: wing 4, ribs 5, wing 4, to form a left wing assembly.

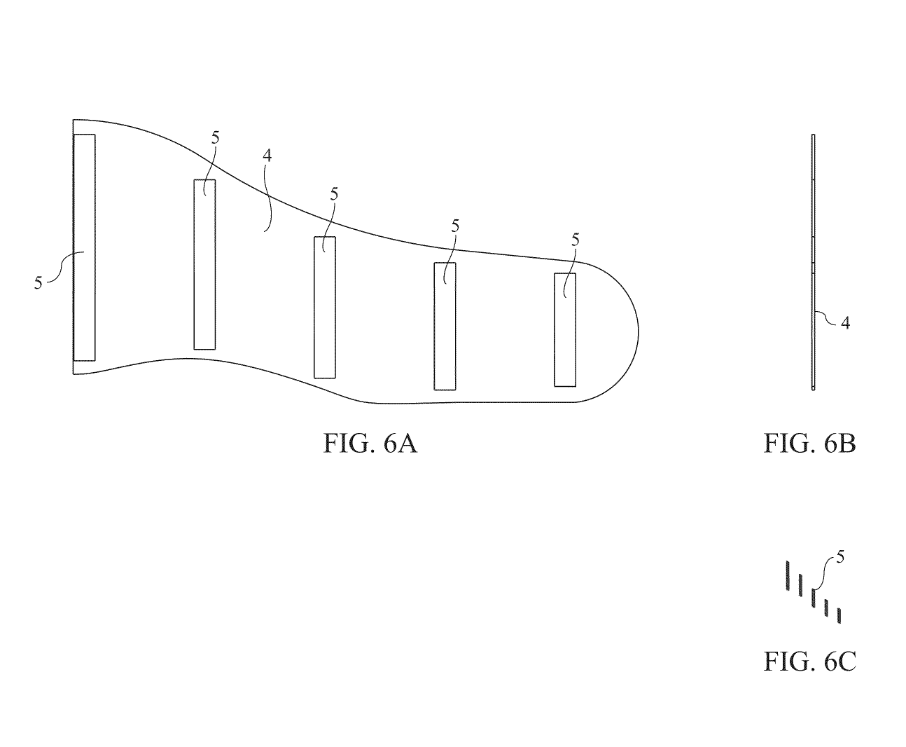

FIGS. 6A and 6B depict one of the wings 4 with multiple ribs 5 thereon without the corresponding second wing 4 over the top. The ribs may be for example essentially orthogonal with respect to the length of the wings 4 (as shown in FIG. 6A), or the ribs may be in another configuration that is suitable for providing the desired support. By way of non-limiting example, the ribs may be substantially parallel to one another (as shown in FIG. 6A), or they may be in different angles with respect to one another, and they may optionally cross one another.

The present devices include arms that emerge from slit in the wings, which are held together and made adjustable through use of a corset pulley system assembly (or sub-assembly) to which the arms are connected. A user may pull the arms for attachment over or around their belly and the arms and corset assembly provide elasticity and tension such that when the arms are attached to one another, the device provides adequate support to the user.

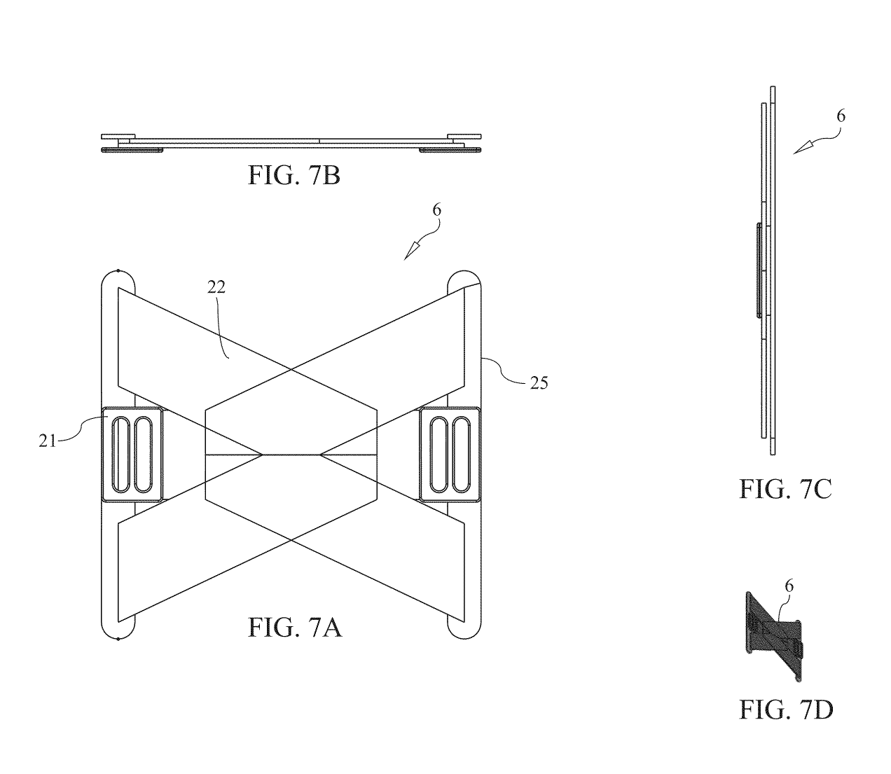

A non-limiting example of the corset pulley assembly (or sub-assembly) enclosed within the overall support device of the present invention, examples of which are depicted e.g., in FIGS. 1 and 2, is depicted in FIG. 7A. The corset assembly includes, inter alia, multiple pieces of corset fabric 22, which may be formed for example, in an overlapping “V” formation, but other formations or configurations are contemplated. The corset assembly may also include clips 21 or other fasteners for connecting arms and enabling tightening and securing of the arms upon use of the device. The corset assembly may also include and corset bones 25 on either side of the corset assembly. The clips may be formed for example of a plastic or metal, but may be any suitable material. Similarly the corset bones 25 may be formed from any suitable material that may assist in maintaining the shape, rigidity and support of the corset assembly such as rigilene boning. The clips may be other than the configurations shown, so long as they achieve the goal of attaching the arms in an adjustable fashion.

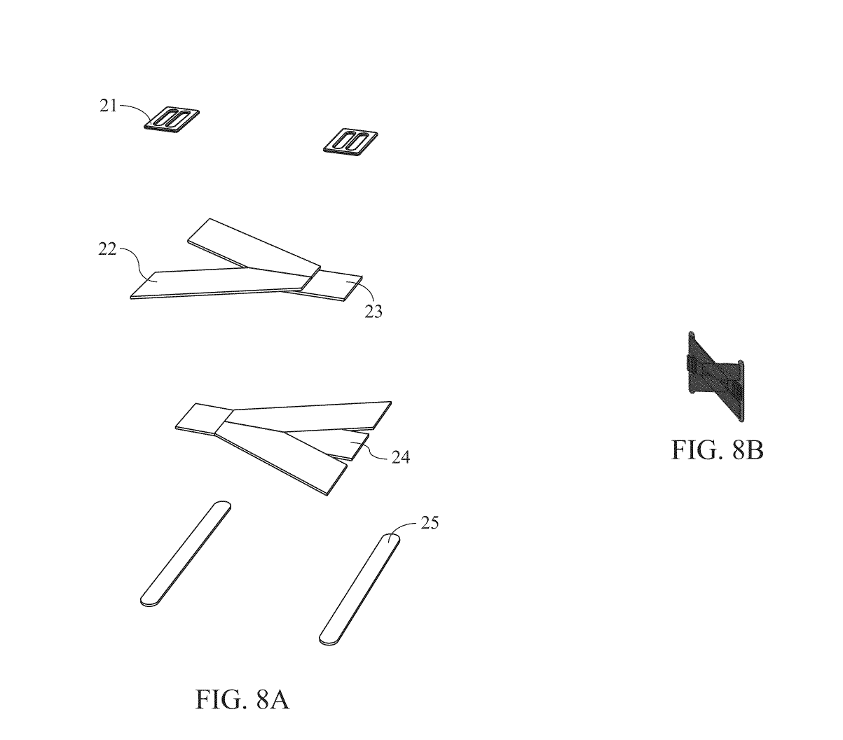

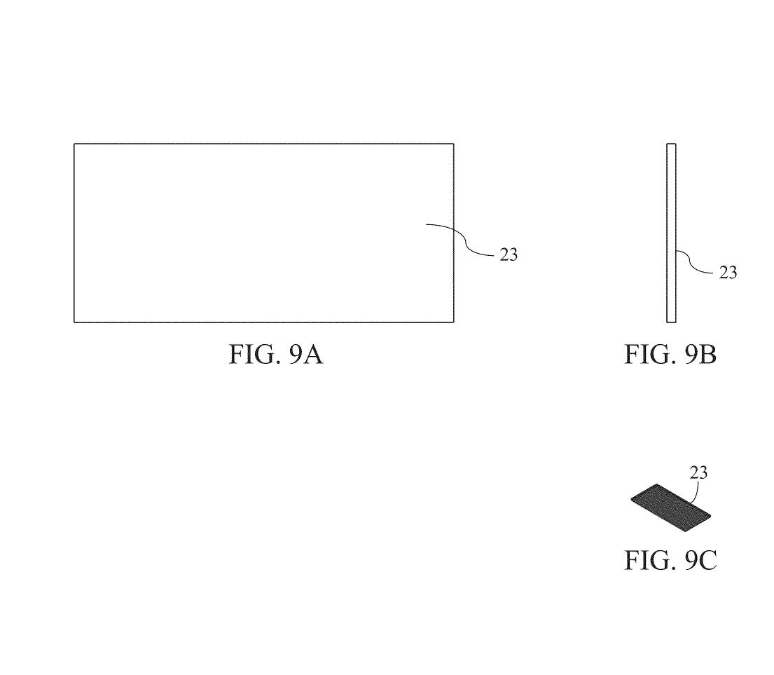

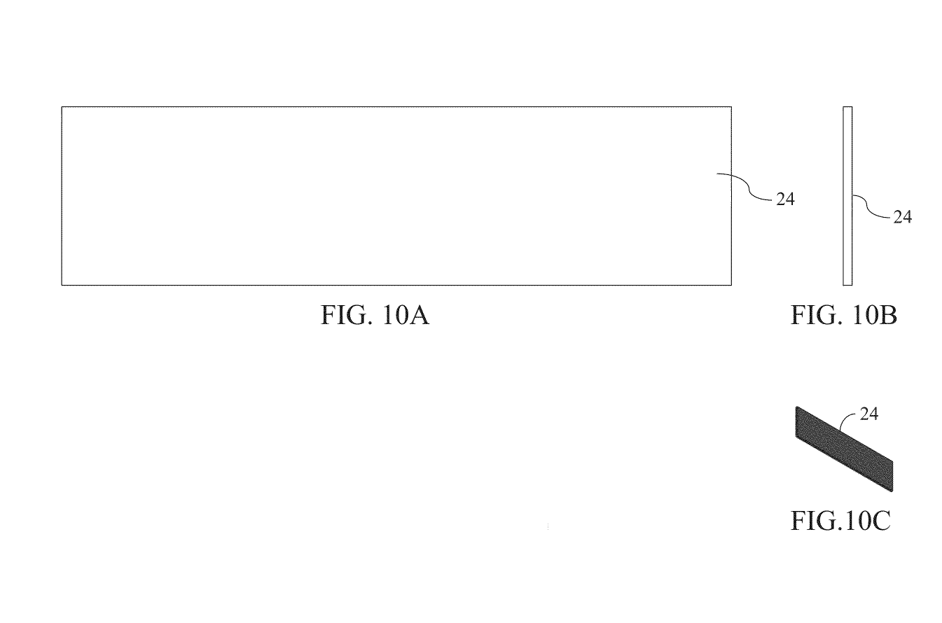

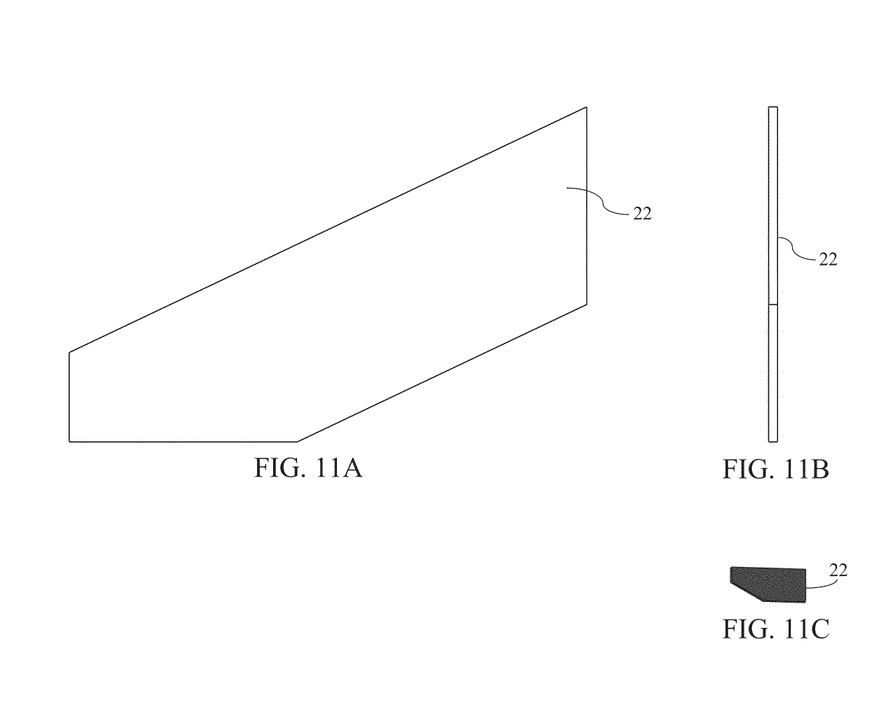

The corset pulley system assembly of FIG. 7A is further shown in FIG. 8 in an exploded view. As depicted in FIG. 8A, the corset assembly may have multiple pieces of fabric 22, 23, and 24 which overlap with one another. The fabric may be formed e.g., of elastic band or other elastomeric or stretch material. The elastic fabrics may be formed for example, into two fabric structures that overlap with one another as depicted. The clips may be configured so that they may attach the corset assembly to the arms. The boning is configured in size shape and placement for support. The fabric structures may include for example two diagonal fabric pieces each to form a “V” shape, and a second fabric piece (23 and 24) to further support holding the corset assembly together and provide further support when the device is in use. Corset fabrics 23 and 24 of a corset assembly according to FIG. 8 are shown in FIGS. 9A and 10A. FIG. 11A shows a diagonal corset fabric portion of a corset assembly according to inter alia, FIG. 8A. According to non-limiting embodiments, two such diagonal portions 22 along with a second corset fabric (23 or 24) form the two fabric structures which overlap with one another in the corset assembly of the present devices. According to alternative embodiments, one or more of the fabric pieces 22, or pieces 22 and 23, or pieces 22, and 24 may be formed from a single piece of fabric, rather than from separate pieces of fabric sewn or otherwise attached to one another. The elements of FIG. 8A may be for example, as follows:

| ITEM NO. | PART NAME | MATERIAL | QTY. |
|---|---|---|---|
| 21 | CLIP | ABS PLASTIC | 2 |
| 22 | CORSET FABRIC 3 | ELASTIC BAND | 4 |
| 23 | CORSET FABRIC 1 | ELASTIC BAND | 1 |
| 24 | CORSET FABRIC 2 | ELASTIC BAND | 1 |
| 25 | CORSET BONE | REGILENE BONING | 2 |

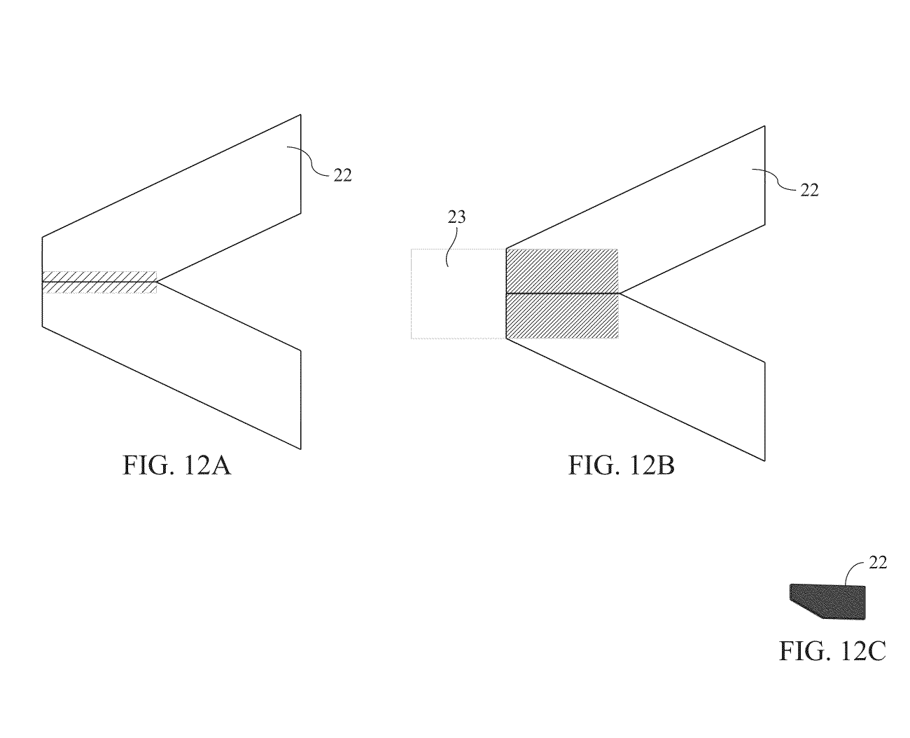

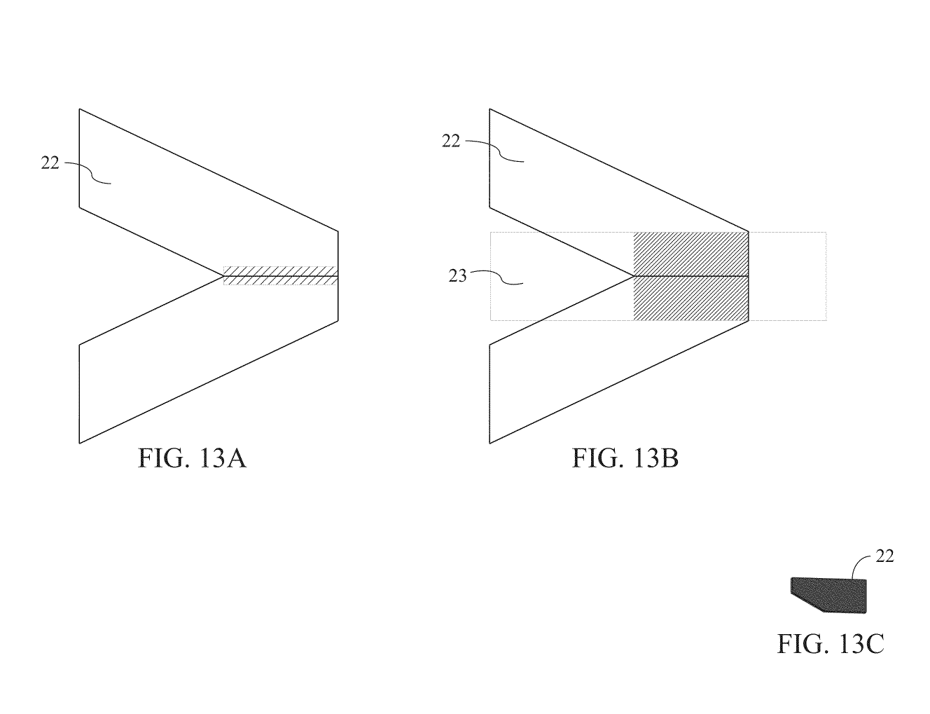

FIGS. 12A and 12B further depict one side of the corset assembly in accordance with non-limiting example embodiments, and in particular, the stitching of fabrics together along the hatched area. FIGS. 13A and 13B further depict the other side of the corset assembly in accordance with non-limiting example embodiments, and in particular, the stitching of fabrics together along the hatched area. The elements of FIGS. 12A and 12B may be for example, as follows:

| ITEM NO. | PART NAME | MATERIAL | QTY. |
|---|---|---|---|
| 22 | CORSET FABRIC 3 | ELASTIC BAND | 4 |
| 23 | CORSET FABRIC I | ELASTIC BAND | I |

The elements of FIGS. 13A and 13B may be for example, as follows:

| ITEM NO. | PART NAME | MATERIAL | QTY. |
|---|---|---|---|
| 22 | CORSET FABRIC 3 | ELASTIC BAND | 4 |
| 23 | CORSET FABRIC 2 | ELASTIC BAND | 1 |

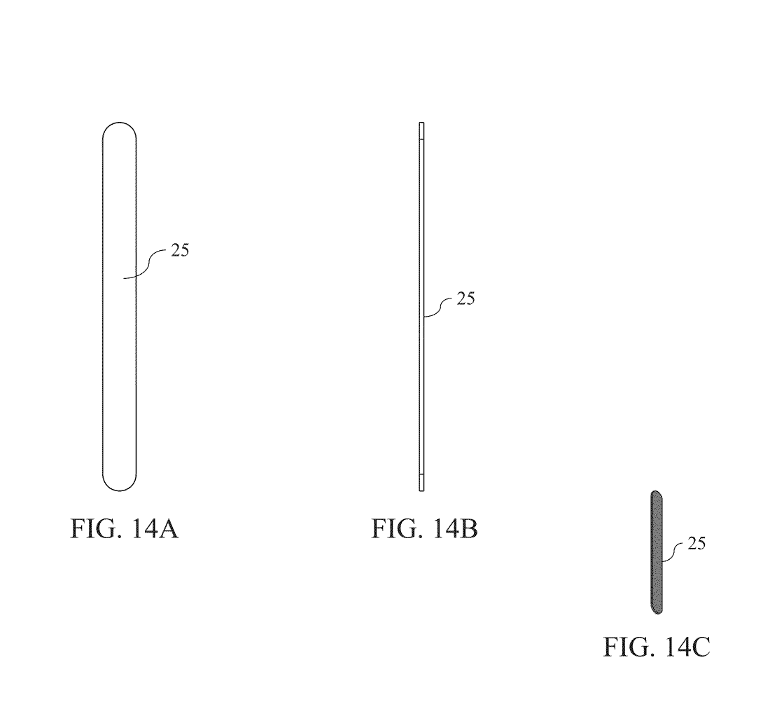

FIGS. 14A and 14B depict a corset bone of a corset assembly according to inter alia, FIG. 8A. As indicated above, the corset bone may be on either side. As indicated above, the correct bone may be on either side of the corset assembly 6, and may be made of e.g., rigilene boning.

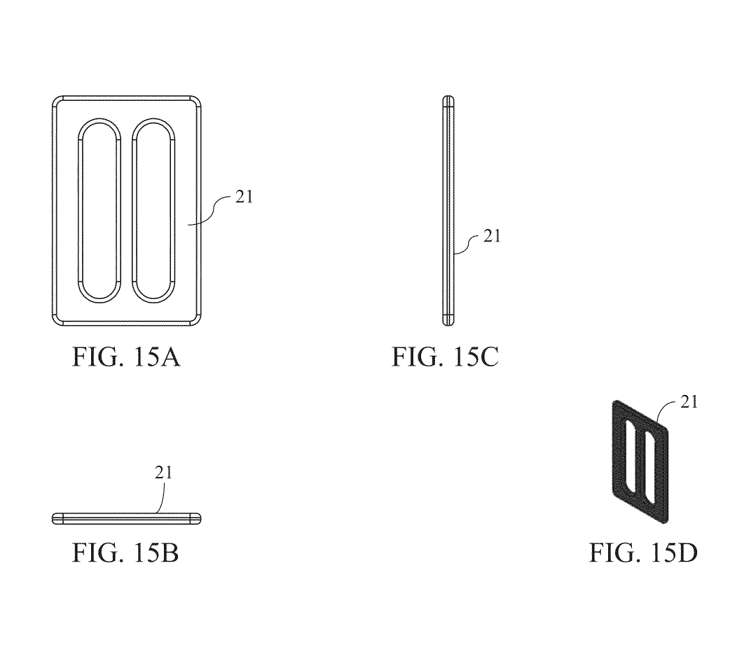

FIGS. 15A-15C depict views of a clip of a corset assembly according to inter alia, FIG. 8A.

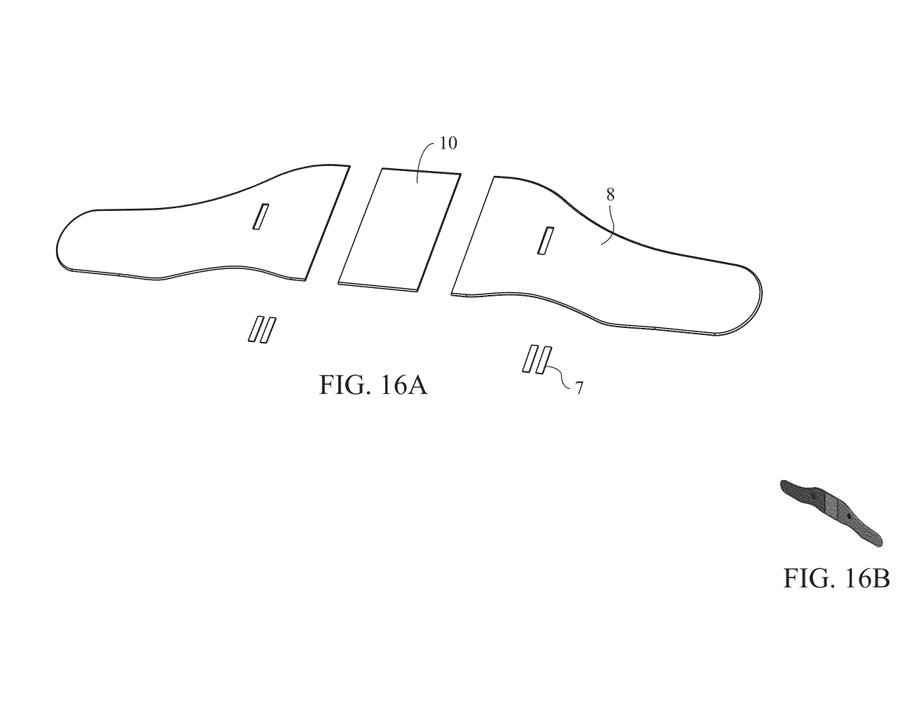

FIG. 16A depicts a front perspective exploded view of a wing assembly according to non-limiting embodiments of the lumbar device of FIGS. 1A and 2. In particular, FIG. 16 depicts first and second wings 8, which include slits adapted in size and shape for the first and second arms to pass through respective slits. The slits may be reinforced e.g., with slit bones on an underside of the slits. FIG. 16A also shows that the first and second wings may be attached by one or more fabric portions 10. According to the non-limiting example depicted in FIG. 14, the wings 8 are attached with a cotton center fabric 10 One or multiple pieces of fabric may be present and the device is not limited to the depicted configuration. The wings 8 may be made e.g., of fleece, for comfort, although other materials are certainly contemplated and are included herein.

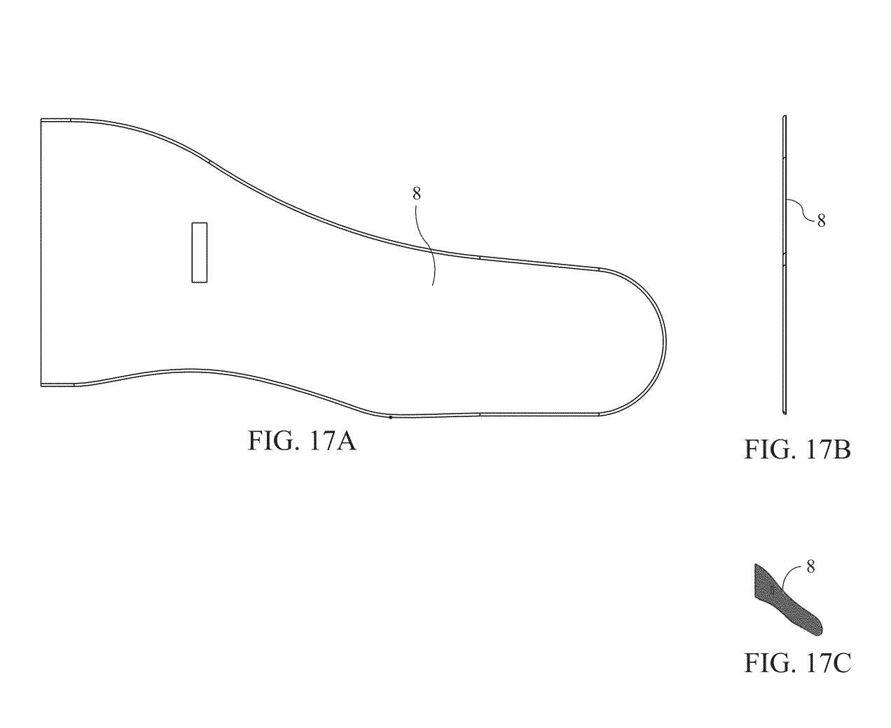

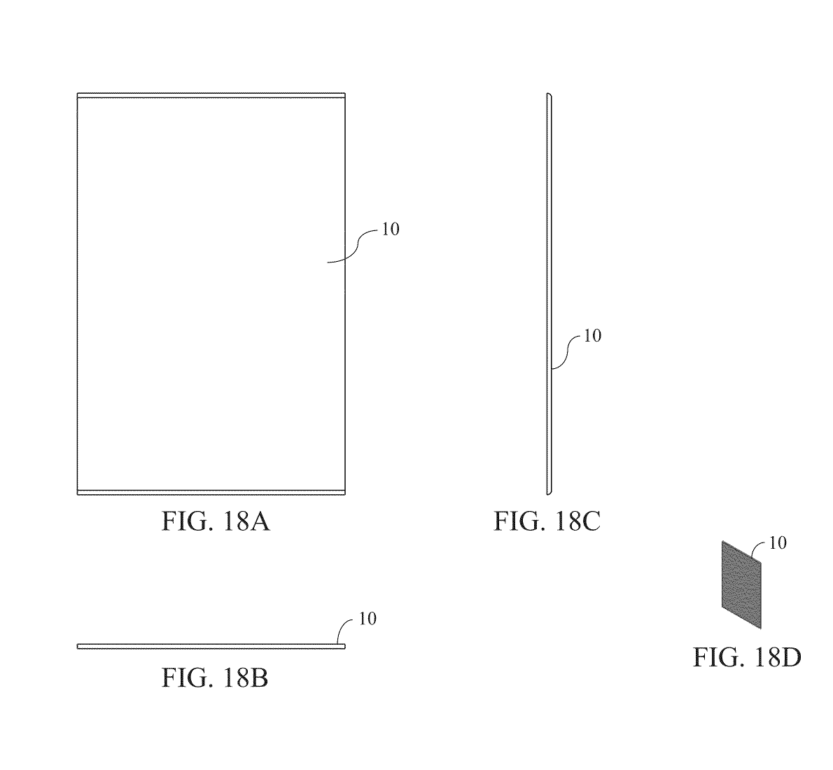

One of the wings 8 from e.g. FIG. 16 is depicted in FIG. 17A, which shows the slits through which a respective arm may pass. FIG. 18A depicts a piece of fabric 10, which is a piece of fabric between the wings 8. Multiple pieces of fabric 10 may be used. This fabric may be made e.g. of cotton.

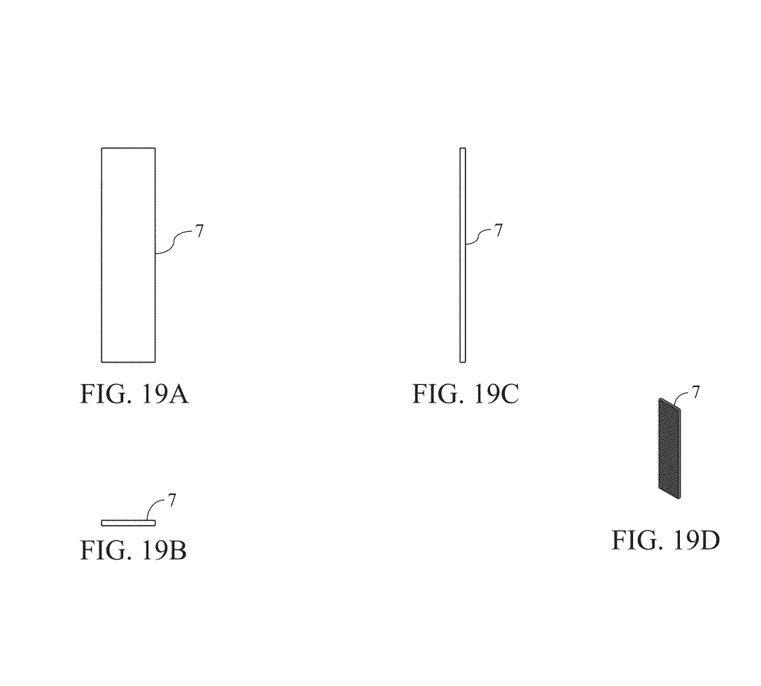

FIG. 19A depicts a slit bone 7, which may be made e.g. of rigilene boning or other suitable material.

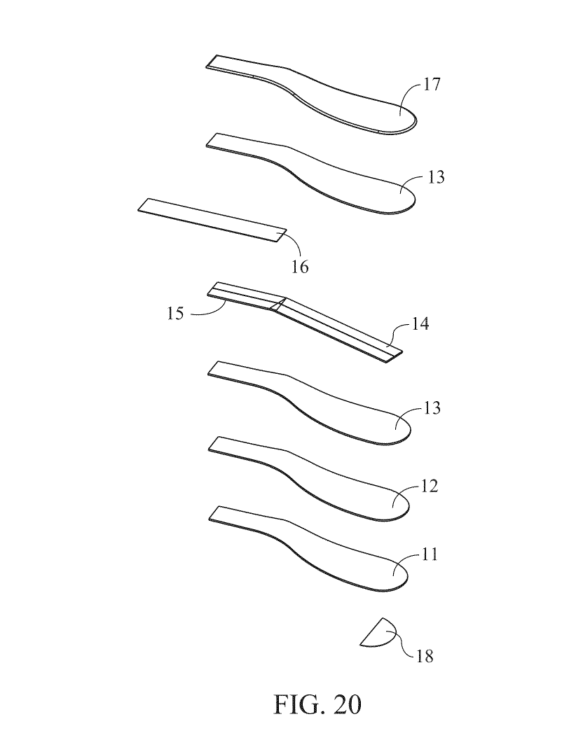

The arms of the present devices may be formed from many different layers, as shown e.g., in the exploded view of FIG. 20. By way of non-limiting example, arms of the present devices may include for example, one or more cotton arms 11, one or more arm padding 12 layers, one or more a mesh arm layers 13, arm bones 14 and 15, an arm strap 16, which may be elastic, a fleece arm 17 and (or other fastener). One or more cotton layers 11, one or more padding layers 12, one or more mesh arm layers 13 (two layers are shown), arm bones or boning layer (14 and 15) and an elastic arm strap 16. Embodiments of FIG. 20 have the following components.

| ITEM NO. | PART NAME | MATERIAL | QTY |
|---|---|---|---|
| 11 | BLACK COTTON ARM | COTTON | 2 |
| 12 | ARM PADDING | COTTON | 2 |
| 13 | BLACK MESH ARM | MESH | 4 |
| 14 | ARM BONE 1 | RIGILENE BONING | 4 |
| 15 | ARM BONE 2 | RIGILENE BONING | 4 |
| 16 | ARM STRAP | ELASTIC BAND | 2 |
| 17 | BLUE ARM | POLAR FLEECE | 2 |
| 18 | ARM VELCRO | VELCRO | 1 |
| 21 | BLUE SQUARE | | 2 |

As explained herein, the arm attachment 18 (which is Velcro® depicted in FIG. 20), may be substituted by any removable or detachable attachment for fabric known to those skilled in the art. The arms 17 may be attachable to each other when the support device is in use, by an attachment on one or both of the arms 17, including but not limited to a clip, snap, hook, Velcro® and/or zipper. In the case of Velcro® (hook and loop fasteners), the Velcro® may be attached to one or both of the arms 17, as the material. In example embodiments, a hook portion of Velcro® is attached (e.g. sewn or glued) near the end of one of the arms, and a loop portion of Velcro® is attached near the end of the other arm 17.

It is also possible that only one arm has Velcro® attached thereto (the hook portion), if the arms 17 are made of a material to which the hook portion would attach upon contact therewith.

Although labeled number 17 in e.g., FIGS. 1 and 2, the arms include multiple layers (e.g., 11-17) and components such as those set forth in the exploded view of FIGS. 2 and 20.

The arms include an outer layer 17, which may be made e.g. of fleece. Those skilled in the art would recognize that different materials may be used for each of the layers. For example, the cotton layers and padding may be replaced with other suitable materials, which preferably may provide comfort, cushioning, breathability and/or other advantageous features, as would be apparent to those skilled in the art.

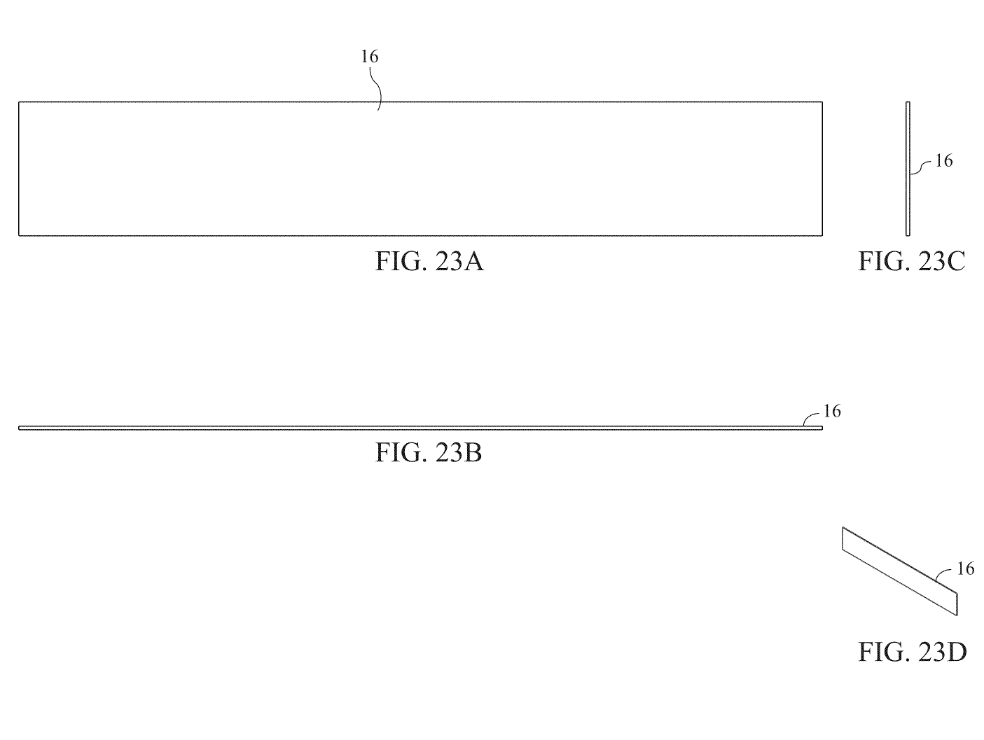

FIG. 23C depicts an elastic arm strap 16, which is a component of each arm. The arm strap is a component for comfort and safety of the user, as it prevents the user from over-tightening the strap and device as a whole—which is of particular concern in maternity use of the device. The present devices should not be over-tightened to the extent where it becomes uncomfortable and/or restrictive.

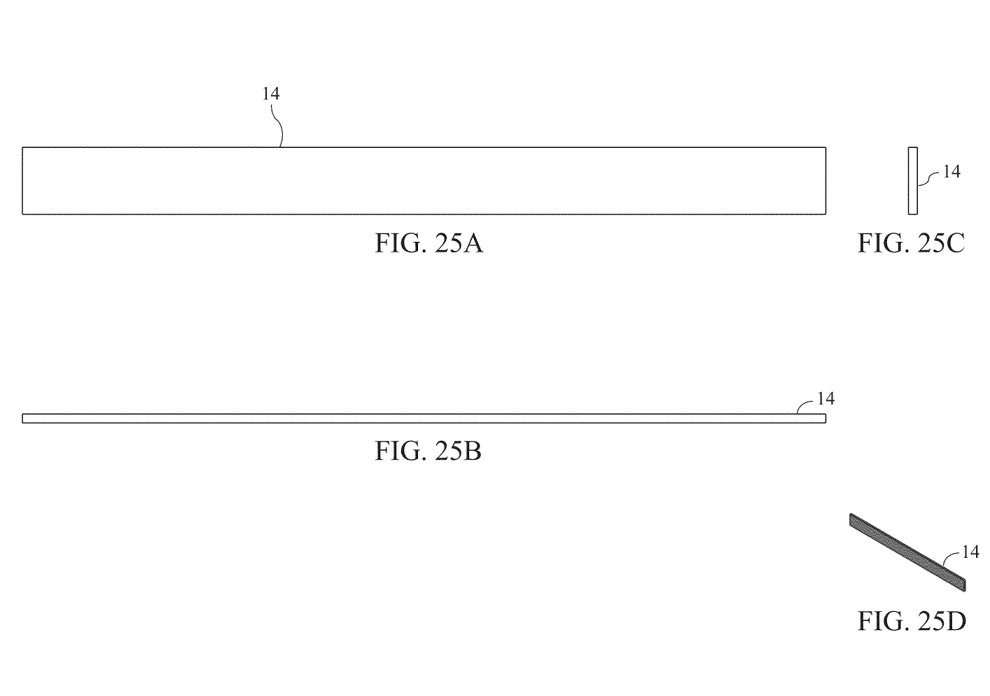

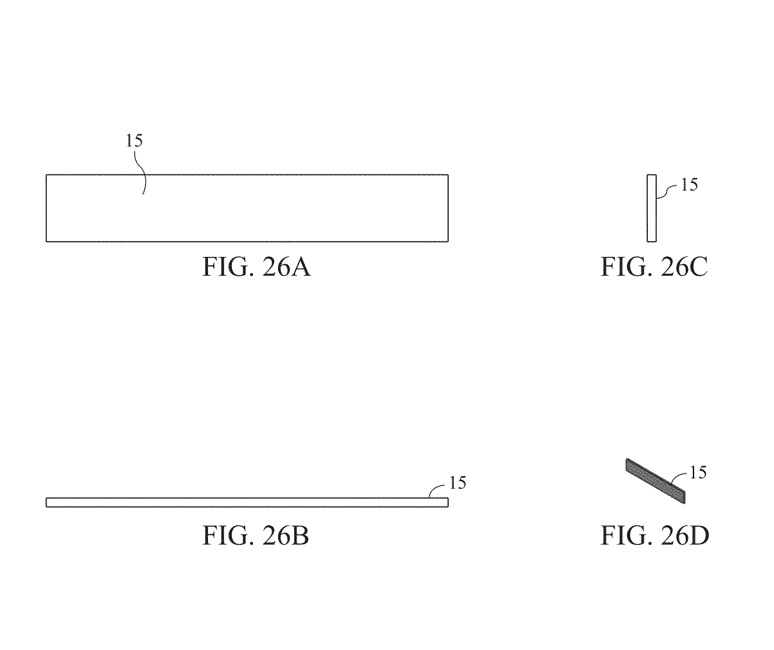

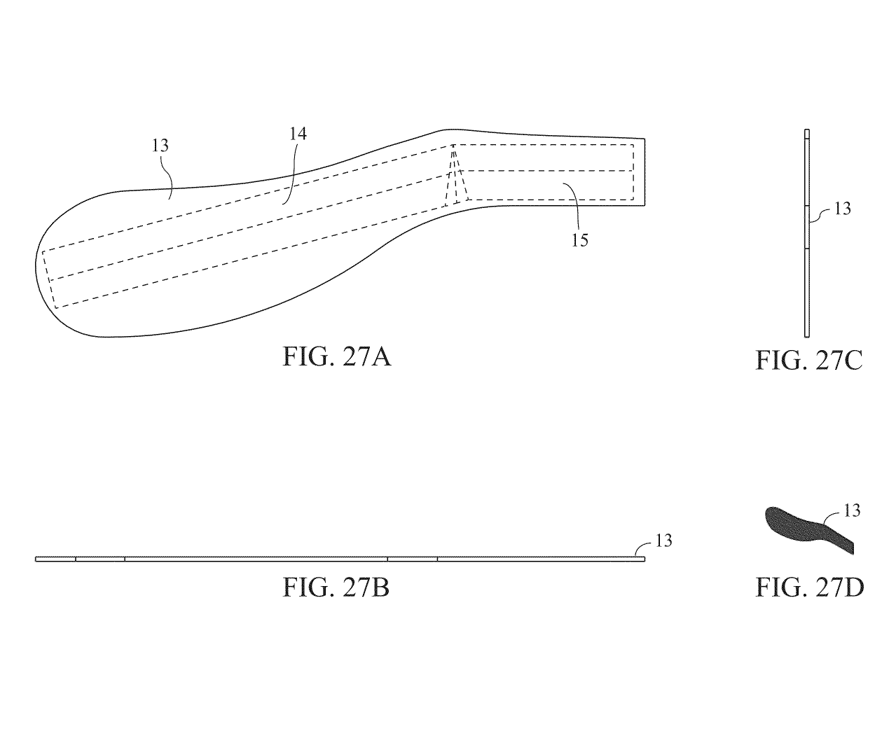

The arm may have multiple bones as depicted forth for example, in FIGS. 25A-27C. FIGS. 25A-D show a first example bone 14. FIGS. 26A-D show a second example bone 15. FIGS. 27A-D show an example of relative placement of such bones with respect to a mesh arm 13. Embodiments of FIG. 27A-27D have the following components.

| ITEM NO. | PART NAME | MATERIAL | QTY. |
|---|---|---|---|
| 13 | BLACK MESH ARM | MESH | 4 |
| 14 | ARM BONE 1 | RIGILENE BONNING | 4 |
| 15 | ARM BONE 2 | RIGILENE BONNING | |

The boning layers(s) may be made e.g., of rigilene boning, metal, plastic or other material that may provide the required support and rigidity and shape keeping features. The mesh layer is for breathing and may include any suitable type of mesh known in the art. Other breathable materials are contemplated however as well.

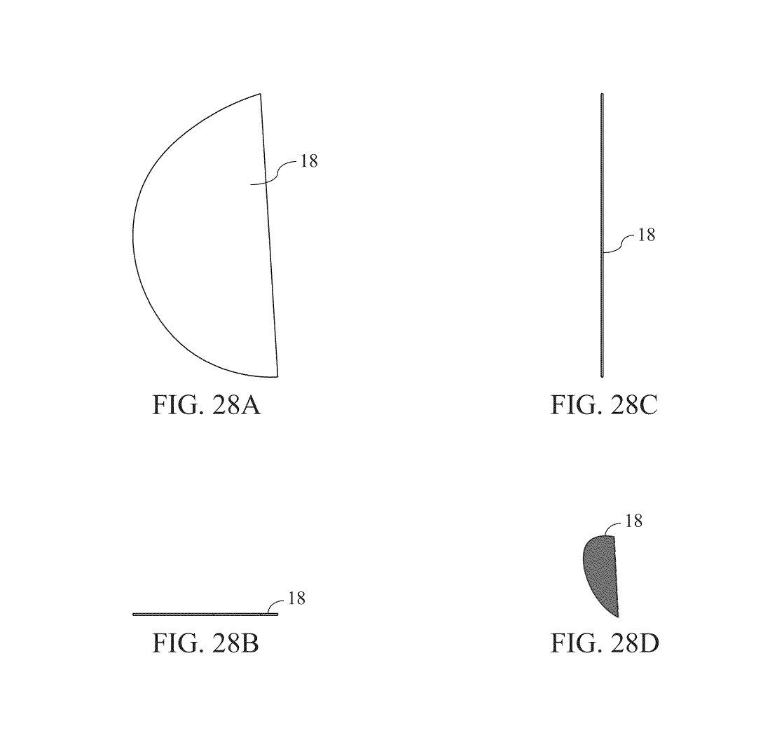

FIG. 28A depicts arm attachment portions 18 (or “arm fasteners”), which may attach one arm to the other arm to secure the arms around, over and/or under the belly of a user. Although the arm attachment 18 is depicted as a hook and loop fastener, e.g., Velcro®, in a somewhat semi-circular shape, (e.g., in FIGS. 2, 19, and 28A), other attachments and/or shapes are contemplated and may be used. The essentially semi-circular shape was selected for the depicted embodiment to roughly correspond to the ends of the arms for secure attachment of the first and second arms to one another. The fastener may be attached to the arms, e.g., by sewing or adhesive or other attachment, which may be permanent or semi-permanent. In particular, according to non-limiting example embodiments, a hook portion of Velcro® may be attached to a front or back of one end of a first arm, and a loop portion of the Velcro® may be attached to the opposite side (front or back) of the second arm, such that when one puts the brace on, and pulls the arms around a desired position with respect to the user's belly, one arm overlaps the other and the hook and loop portions at least partially overlap for attachment to one another upon contact, thus securing the arms over a desired portion of a user's belly.

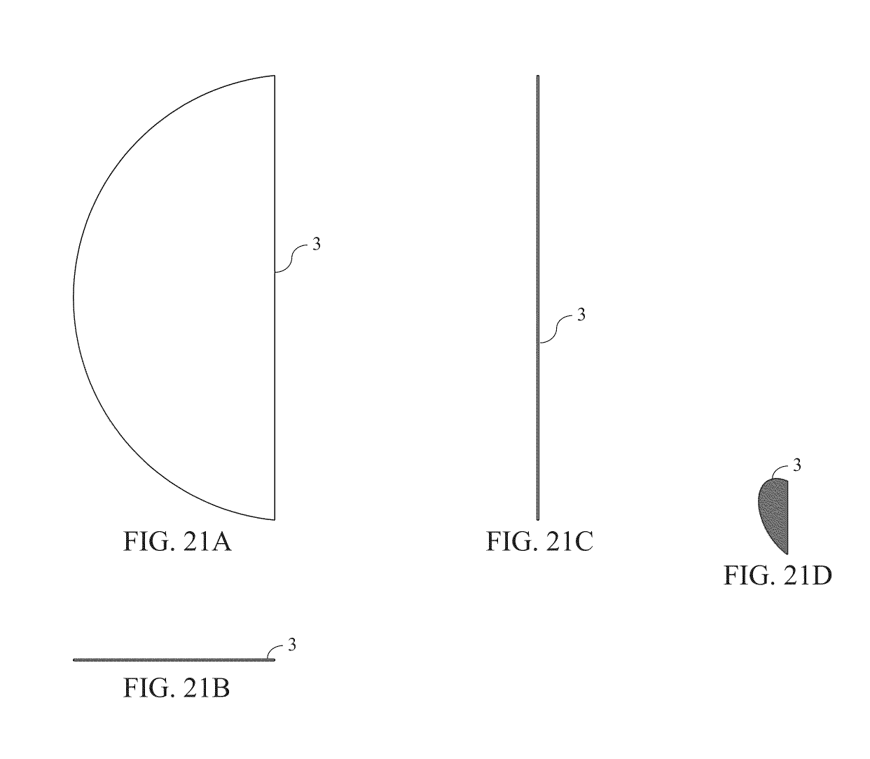

FIG. 21A depicts wing fastener portions 3, (or “wing fasteners”) which may attach one wing to the other wing to secure the wings around, over or under the belly in a desired position. Although the wing fastener 3 is depicted as a hook and loop fastener (e.g. Velcro®) in a somewhat semi-circular shape, (e.g., in FIG. 2), other attachments and/or shapes are contemplated and may be used. The essentially semi-circular shape was selected for the depicted embodiment to roughly correspond to the ends of the wings for secure attachment of the first and second wings to one another. The fastener may be attached to the wings, e.g., by sewing or adhesive or other fastening device, which may be permanent or semi-permanent. In particular, according to non-limiting example embodiments, a hook portion of Velcro® may be attached to a front or back of one end of a wing, and the loop portion of Velcro® may be attached to the opposite side (front or back) of the second wing, such that when one puts the brace on, and pulls the wings around a desired portion of their belly, one wing overlaps the other and the hook and loop portions at least partially overlap for attachment to one another upon contact, thus securing the wings over a desired portion of a user's belly.

By way of non-limiting example, the “female” portion of a hook and loop fastener may be attached to an underside of a right wing and/or right arm and the “male” portion of a hook and loop fastener may be attached to a top side of the left wing and/or left arm that when the straps are folded across a wearer, the “male” and “female” portions come into contact with one another and secure the straps in a desired place/fit. It should be understood however, that the fasteners may be varied and that when hook and loop fasteners are used, the fasteners may be reversed (e.g., “male” on the underside of the wing and “female” on the top of the wing, etc.)

In one or more layers of the device, the first and second wings may be a continuous piece of fabric (see e.g. layers 1 and 2). Other layers of the wings may have the first and second wings formed separately and connected to one another (see e.g. wings 4-mesh) and 8 (fleece). It is contemplated that the first and second arms may include one or more portions or layers that are a continuous material and may contain layers that are separate from one another, e.g., the separate mesh arms and bones. During assembly of the device multiple layers may be pre-attached to one another. The layers depicted e.g., in FIG. 2 do not need to be attached in the order shown. For example, one may assemble e.g, the straps, corset pulley system assembly and/or wing/rib assembly separately, before assembling the entire device.

Although a particular shape and size of the brace device herein and components thereof are depicted, other shapes and sizes may be suitable as well. For example, the brace may come in various sizes for differently sized people (by height and or weight). The brace may come in for example, small, medium, large, and extra large sizes. By way of non-limiting example, the sizes may be for belly sizes within a particular range. For example, size Small may be 28.5-43.1 inches, Medium may be 30.4-47.1 inches, Large may be 33.3-52.4 inches, and Extra Large may be 36.5-57.6 inches. Belly size may be determined by taking a circumferential measurement of the patient's waist, starting from the hips to the umbilicus. The appropriate size may also be determined by taking into account how far into the pregnancy a user is. For example, if it is early in a pregnancy, a user may wish to obtain a brace that is one size larger than the measurement indicates.

The device may also be shaped in any way so as to provide a suitable fit to a wearer. By way of example, the example device depicted in FIG. 1C, has a bottom shape that is not straight across, but rather has two curved indents at the portions that may correspond to the location of hips of the wearer. According to non-limiting embodiments, the bottom shape may be straighter or have a greater curvature than that depicted.

The present devices may also include at least one panel fastener configured for attachment of at least one padded panel to the device. According to non-limiting example embodiments, a panel fastener may include e.g., one side (e.g., loop) of a hook and loop fastener.

The lumber support devices themselves may further include at least one detachable/removable padded panel selected from an anterior panel, a posterior panel and a coronal panel.

The at least one removable padded panel may be attached to the device by at least one panel fastener. That is, the panel may have e.g., an opposite portion of a hook and loop fastener from a portion included on the main portion of the device.

According to example embodiments, the at least one panel may be configured with an opening to attach the panel to the device by sliding the panel onto the device. Thus, the panel may have e.g., loops or straps.

FIGS. 29-34, 41-42, 44-46B and 48A and 48B depict non-limiting examples of optional, removable padded, but rigid anterior, posterior and coronal panels, which may be added or removed from the present devices by a user. In particular, one or more panels may be desired for use in a post-natal kit, or in non-pregnancy uses. Under a doctor's supervision, one or more panels may be used during early pregnancy, but in no event should the attachable panels be used during late stages of pregnancy.

FIG. 29 depicts a front view of a removable padded posterior panel 26 in accordance with non-limiting example embodiments. As shown in FIG. 29, the edges 27 may be padded with e.g., soft foamy plastic. The front 28 may be added with foam to soften over hard plastic. Additionally, the posterior panel may have multiple holes 29 to allow breathability of the panel. Example embodiments of the panel may be configured such that the top of the panel reaches e.g. the T9 vertebrae (To comply with brace codes L0627, L0631 and/or L0637).

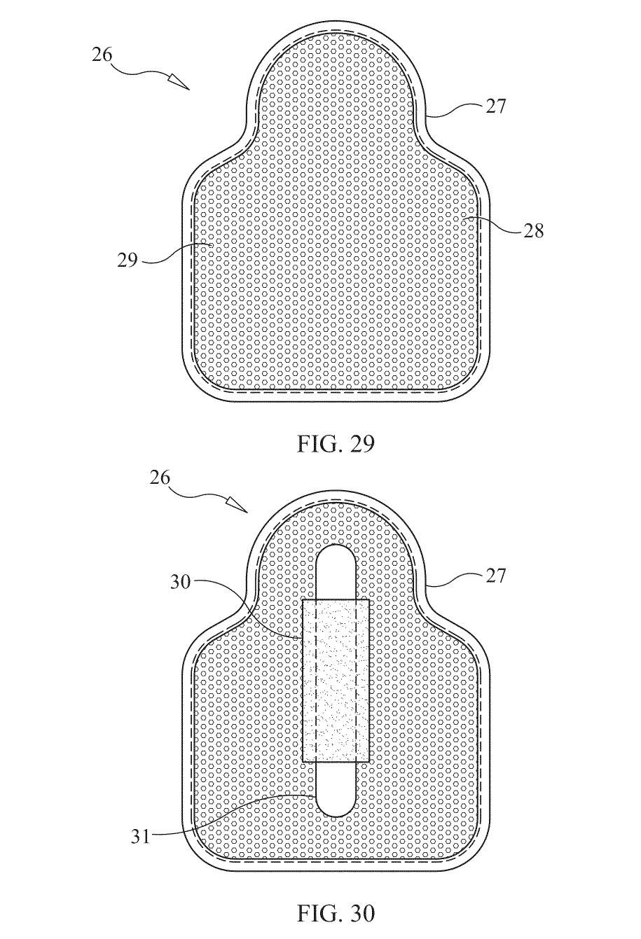

FIG. 30 depicts a back view of the removable padded posterior panel of FIG. 29. As depicted in FIG. 30, the back of the posterior panel may include Velcro® with a removable sleeve (e.g., cloth) configured for the brace to pass through. The posterior panel may be designed e.g., to extend from T9-S1 on the patient. Addition of anterior and posterior panels will advantageously meet the guidelines for L0631 brace code. Posterior panel can also be combined with removable side flanges to enable circumferential coronal and sagittal support and will then meet the guidelines for L0637 brace code used in the USA. Panels can attach e.g., via Velcro® strips and/or Velcro® strip 30 with cloth sleeve 31. According to example embodiments, panels will strictly be used post-partum or in very early pregnancy before any “belly” develops. The posterior panel will have a pillow-like padding covered in cloth on its anterior side as this will rest against the patients' lumbar region from T9-S1.

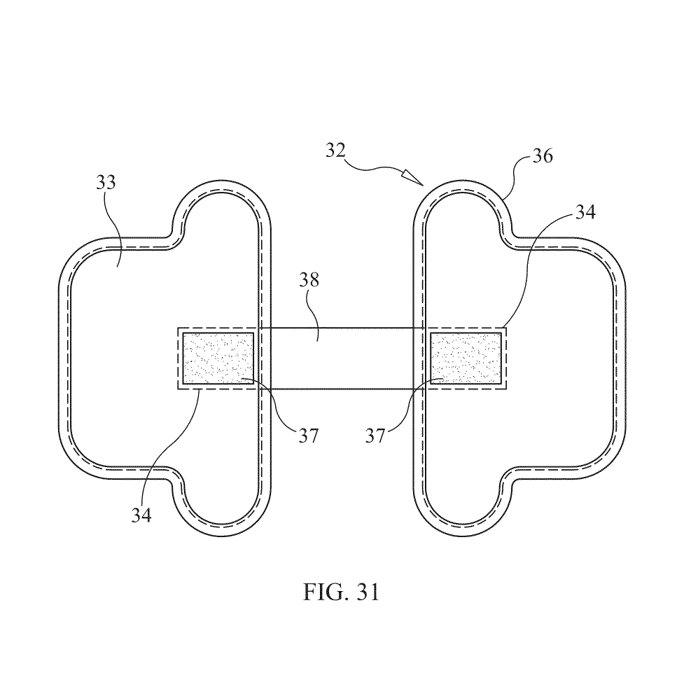

FIG. 31 depicts a front view of a removable coronal side panel (front 32) in accordance with non-limiting example embodiments. FIG. 31 also depicts a smooth cloth 38 layer between the Velcro® attachments 37. Panels 33 may be rigid plastic panels with extra padding on the front side.

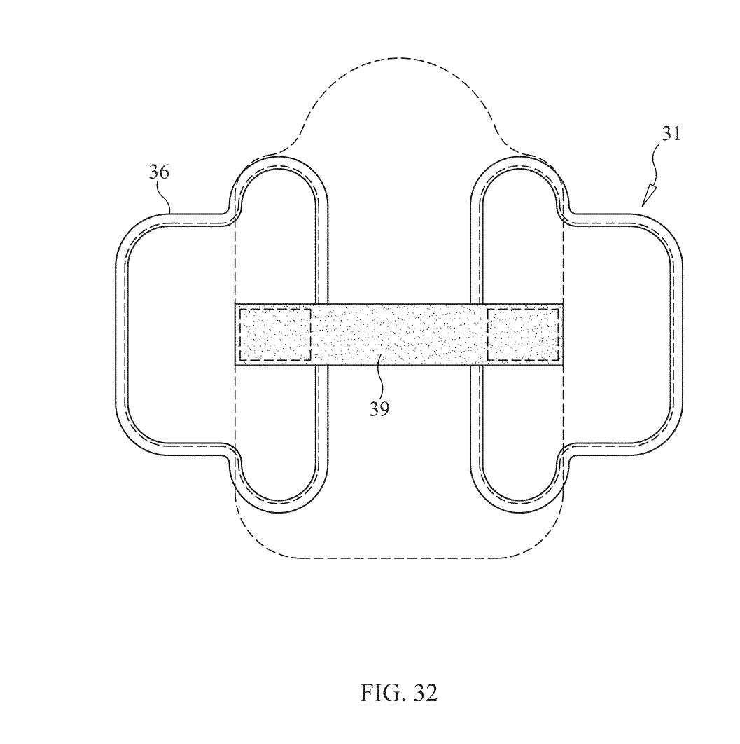

FIG. 32 depicts a back view of the removable coronal side panel of FIG. 31. Coronal Panels (back 31) may have rigid plastic 33 covered with significant extra pillow like padding 34 that may have a cloth covering for comfort. The edges 36 will also have foam like plastic covering and padded edges for comfort. The coronal panels will attach to the anterior aspect of the large Posterior T9-S1 panel. The attachment may be e.g., via Velcro® 39, which faces the posterior. The dotted line in FIG. 32 is where the posterior panel may be located.

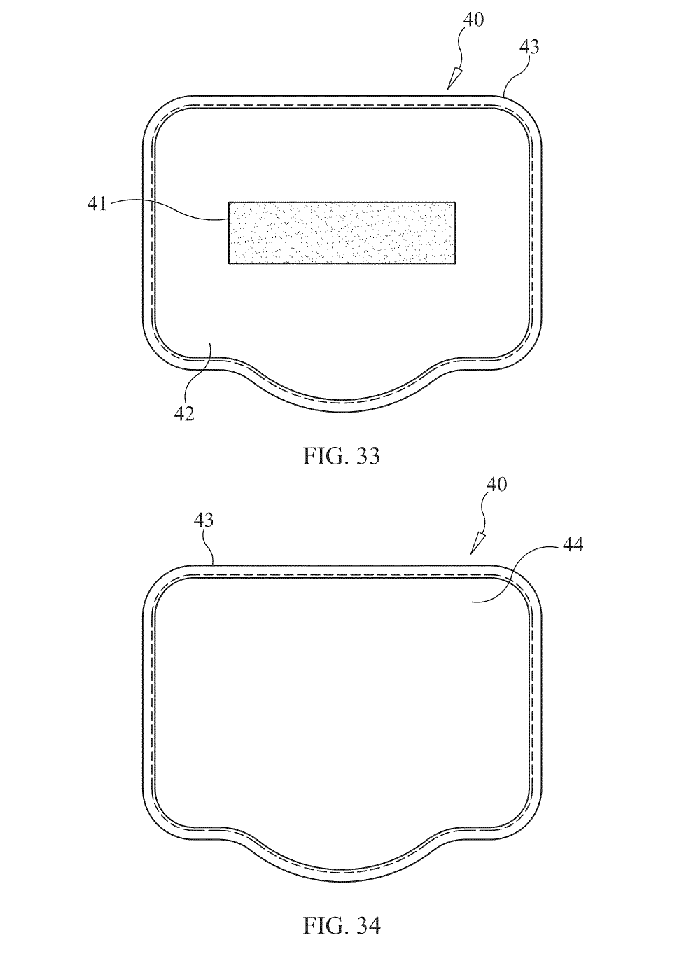

FIG. 33 depicts a front view of a removable anterior panel 40 in accordance with non-limiting example embodiments. The panel in FIG. 33 has padded edges 43, removable Velcro® 41 to attach to the brace, and is made of plastic 42 covered in breathable cloth.

FIG. 34 depicts a back view of the removable anterior panel of FIG. 33. The Anterior panels may have removable Velcro® attachments 41 to the brace itself or can attach via a Velcro® cloth Sleeve. The anterior panel may have a pillow-like padding covered in cloth. This will face posteriorly as it will rest against the abdomen. The anterior panels have padded edges 43 and extra padding 44.

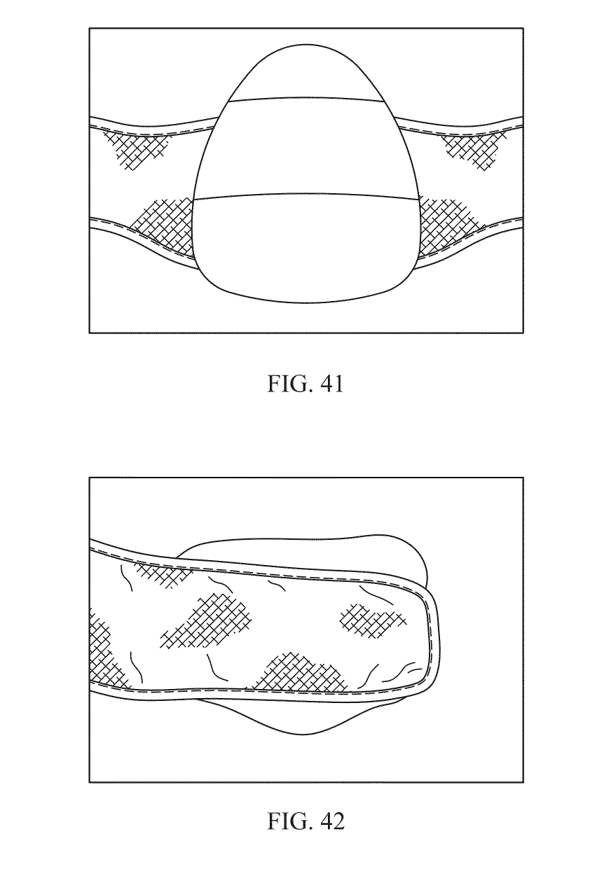

FIGS. 41-42 and 44-46 depict panels of padding in accordance with non-limiting example embodiments of the present invention.

As discussed herein, additional padding and/or supports may be added to the support devices of the present application. By way of non-limiting example, removable padded panels may be added to the back of the device, e.g., posterior panels as shown in FIGS. 41 and 46, or the front (belly) portions of the device, e.g., anterior panels as shown in FIGS. 42 and 44-45, which may be removably attached to the device, e.g., by attachments, which may include e.g., one or more of the following: Velcro®, clips, snaps, straps or other connector that permits removal of the padding. Thus, example embodiments further include one or more attachments. Further example embodiments include one or more padded panels.

The present devices are advantageous in that they are the only known maternity braces that can be used throughout an entire pregnancy AND post-partum, because of the modular nature of the device, and use of padded, but rigid, anterior, posterior, and/or coronal panels. Additionally, the present devices also have a unique pulley system that has a unique pulley system with over-tensioning protection. In particular, the elastic in the tightening arms provides over-tensioning protection and also the arms to be positioned in multiple configurations on the pregnant belly.

As indicated herein, optional anterior, posterior, and/or coronal panels may be included with, or added to the present devices. This is the only brace that is so modular that it can be used throughout the entire pregnancy and post-partum. The combination of the unique pulley/elastic, over-tensioning prevention system, the adjustable arms, and the extra padded ridged anterior, posterior and/or coronal panels allow for this modularity.

The optional padded panels allow for reduction in back stresses of the present devices in very early pregnancy and post-partum by using a unique pulley and tensioning system of the present devices and removable panels. All other braces on the market focus on reducing belly stress after the pregnant woman gained weight.

The panels may include e.g., a padded posterior panel that may be primarily used in very early pregnancy until the pregnant woman forms a belly and also post-partum. The posterior panel(s) are not limited by its material. According to non-limiting example embodiments the posterior panel(s) are rigid, but made of a material that will conform to the lumbar region of the person to whom the device is to be applied. Example panels may include one or more materials, such as cloth material, which may be lightweight for comfort and/or one more stiffer or reinforced materials for added stability. Example support materials that may be included for example in the posterior panel may include inner light weight (cloth) with outer-laced mesh to maximize comfort and breathability while at the same time maintaining support. The posterior panel(s) may in some embodiments include an elastic fiber strands within the material which allow for a form fit with tensioning. A central region of a panel may be made of a breathable cloth fabric to interconnect for example spandex/elastic flexible regions.

The posterior panels may include e.g., curved portions, angles, and/or any other desired shape that achieves the desired results and is not limited to the shape depicted in FIG. 29. Additionally, the size of the posterior panels may be determined based on various factors including for example the size of the intended user of the device. For example, as indicated above, devices of the present invention may be formed into sizes such as Small, Medium, Large and Extra large, or into numerical sizes, which may call for a different size back panels.

Example anterior panels may be constructed of any desirable material and/or shape depending on the intended use of the device. For example, a front/anterior panel may be of a shape, size and material to be able to cover an expanding pregnancy belly. Lycra spandex. An anterior panel should only be used very early in pregnancy within the first few weeks but mainly post-partum. The front panel may also include one or more moveable and/or removable portions, such that a wearer of the device may for example gather the panel under or over the wearer's belly, or remove the panel or a portion thereof from the device (e.g., on hot days or to help conform the device to the woman's belly as it grows larger, or depending on wardrobe considerations). A non-limiting example of an anterior panel which may be added to or included with the present devices, is depicted e.g. in FIGS. 42, 44 and 45.

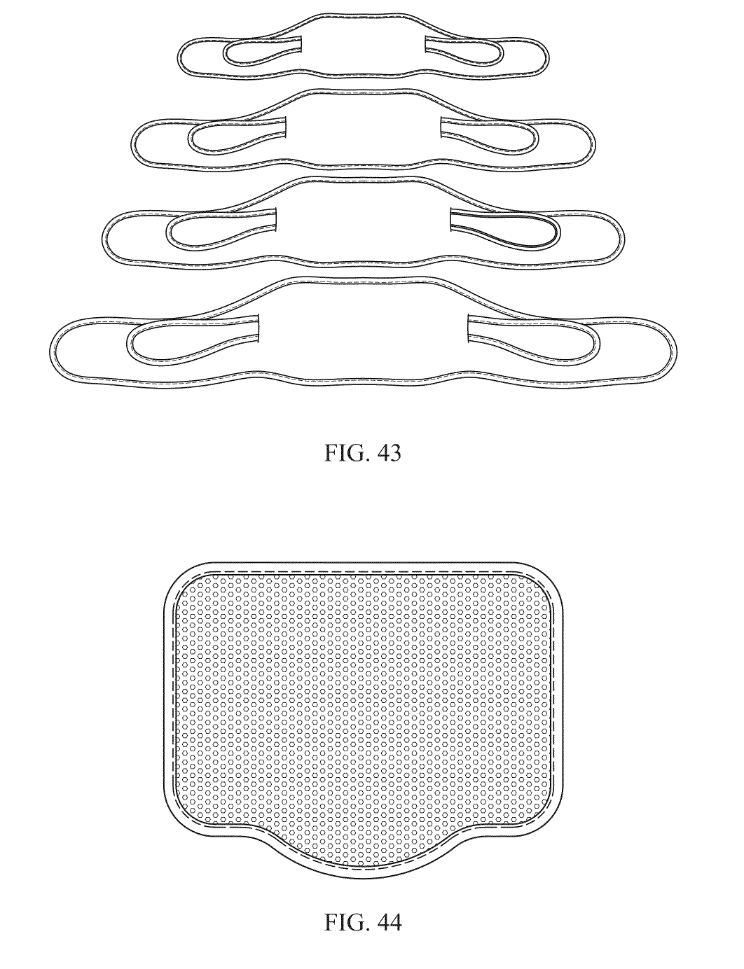

The posterior panel may be e.g., in a substantially rectangular shape. The posterior panel may include curved portions, angles, and/or any other desired shape that achieves the desired results. Additionally, the size of the posterior panel may be determined based on various factors including for example the size of the intended user of the device. For example, as indicated above, devices of the present invention may be formed into sizes such as Small, Medium, Large and Extra large (as shown e.g., in FIG. 43), or into numerical sizes, which may call for different size panels corresponding thereto.

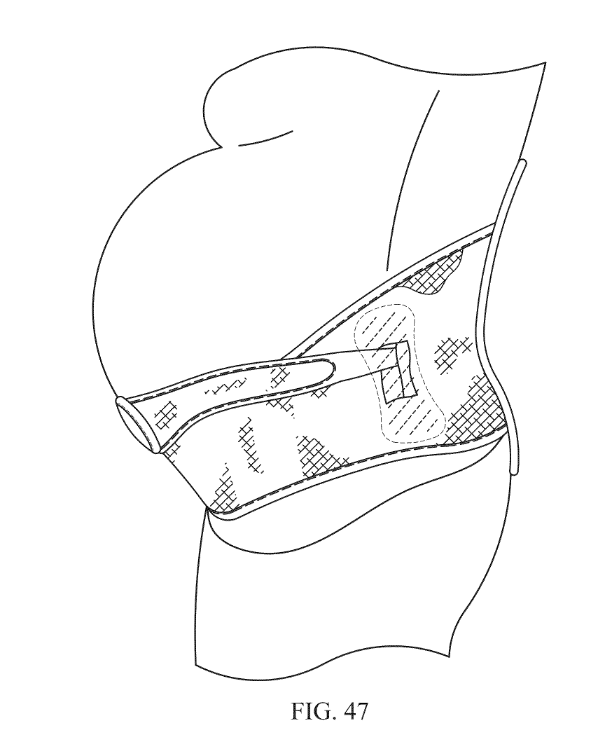

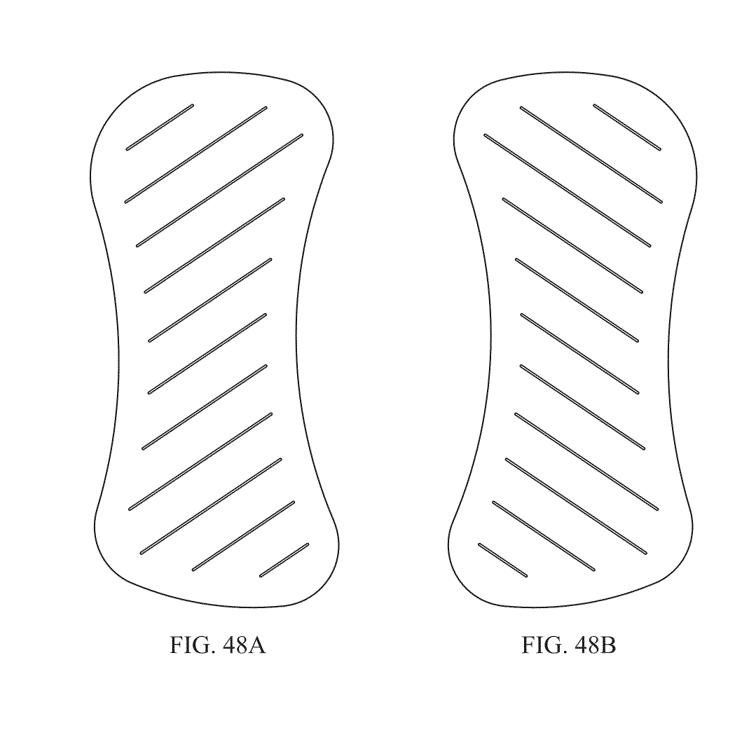

FIG. 47 depicts an example support device/brace in use on an individual, which has coronal panels inserted (slid in) on both sides in the location of the dotted lines on FIG. 47. Non-limiting examples of such coronal support devices are depicted in FIGS. 48A and 48B. As depicted in FIGS. 47 and 48A and 48B, the coronal panels may be inserted on one or both sides. They may include slits to provide air flow. Additionally, the panels may be covered and padded similarly to other panels. The outside may not be padded in certain embodiments. The coronal panels may be made of similar plastic or other materials as the other panels and may be moldable. The coronal panels may be configured such that the device complies with L0637.

Example panels may be constructed of any desirable material and/or shape depending on the intended use of the device. According to example embodiments, anterior, posterior and coronal panels will be rigid but well padded (e.g., with pillow like material covered in mesh cloth), and have multiple holes in the plastic to provide breathability. The front panel may also include one or more moveable and/or removable portions, such that a wearer of the device may for example gather the panel under or over the wearer's belly, or remove the panel or a portion thereof from the device (e.g., on hot days or to help conform the device to the woman's belly as it grows larger, or depending on wardrobe considerations).

The present devices and components thereof may be in any desired color(s). While example colors are set forth in the Figures, these are exemplary only and are not intended to be limiting.

The present devices are the only devices that take into consideration post-partum back pain in C-section patients. The c-section incision cuts through the lower abdominal muscles significantly weakening the patients' core muscles and leading to back pain after delivery. The anterior and posterior panels will reduce back stress and take undue pressure off of the healing muscles which take 6-12 weeks post C-section to heal. The brace is designed to help through all stages of pregnancy and after delivery as well for both C-section patents and vaginal delivery patients.

These are also the only braces that take into consideration post-partum back pain in non C-section patients. In pregnant women that have a vaginal delivery: the lower abdominal muscles are stretched and significantly weakened. This takes weeks to a few months to improve. The patient's core muscles are subsequently weaker and this can lead to back pain after delivery. The anterior and posterior panels will reduce back stress and take undue pressure off of the healing muscles which take weeks to months to heal. Thus, the present devices are That is, the brace is designed to help through all stages of pregnancy and after delivery as well, for both C-section patient and vaginal delivery patients. The anterior panel is designed and specifically pillow padding will prevent excess pressure on the c-section scar. The brace panels should not be used until the wounds are completely healed.

These present devices are modular: allowing the different components such as the panels described herein, to be added or subtracted as a persons' size changes, e.g., to better conform to a women's anatomic changes throughout pregnancy. One of the major concerns with other braces and back supports is that over tensioning can create excess pressure on the abdomen of the pregnant female. The present braces combine multiple safeguards to prevent this (over tensioning) while at the same time providing more contouring support by conforming to the anatomy throughout the changes a female has in each trimester pregnancy.

A goal of the present supports is to provide stabilization throughout each trimester of pregnancy. Initially, during the course of a woman's pregnancy, the corset (e.g., spandex or elastic material) component alone (without the straps attached) may be used during the first and possibly early second trimester, as the straps are typically not necessary to support the belly (which is usually not present into the second and third trimesters). However, as the belly grows throughout the second and third trimester of pregnancy, the arms can be used in a higher position for example in the last days of pregnancy to avoid the brace from sliding off.

Also provided herein are methods of preventing or treating back pain in a person (such as a pregnant, human female or other person who may benefit from support provided by wearing the device) that include applying the devices described herein to a person, such that the person may wear the device. Examples of such devices may include e.g., the devices described herein.

The present methods of preventing or treating back pain in a person, include applying the present lumbar support devices to a person, and securing the device to the person by fastening the wings to one another and then fastening the arms to one another around the user's belly.

The device may be configured such that the fastened arm position with respect to a person to whom the device is applied, is variable.

For example, the arms may be positioned with respect to the person such that the arms are fastened to one another so as to rest at least partially over the fastened wings.

According to other embodiments, the arms may be positioned with respect to the person such that the arms are fastened to one another so as to rest on the upper abdominal belly.

FIGS. 35-40 depict example embodiments of a device according to the present invention and depict the devices in use by various users, in accordance with non-limiting examples.

FIG. 35 depicts a close up view of a portion of a lumbar support device in accordance with non-limiting example embodiments. In particular, this close up view depicts an arm emerging from a slit in a wing of the support device.

FIG. 36 depicts one side of a lumbar support device in accordance with non-limiting example embodiments of the present invention. As shown in FIG. 36, an arm emerges from a slit in the corresponding wing.

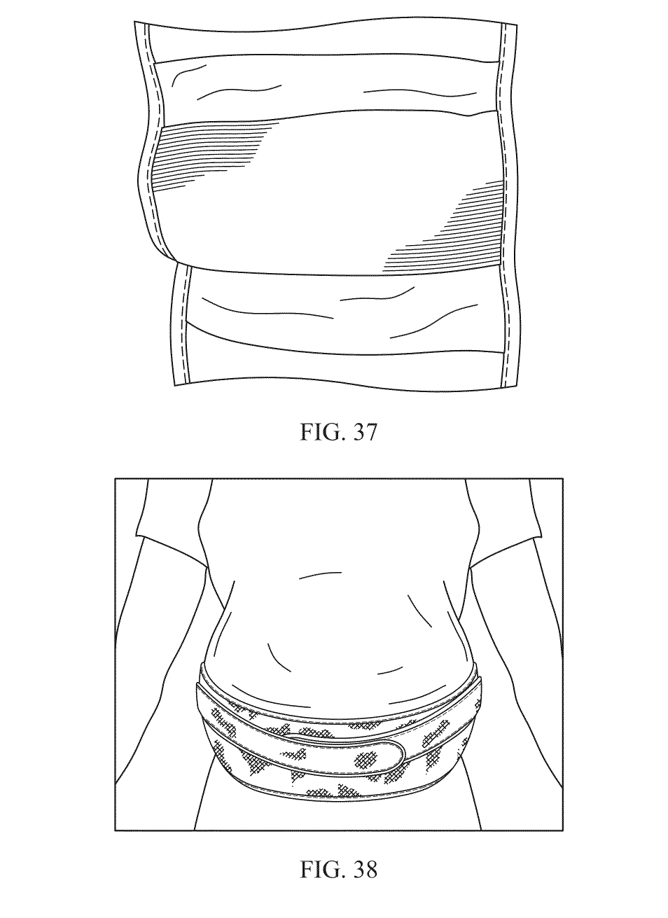

FIG. 37 depicts a middle portion of an underside of the lumbar support device in accordance with non-limiting example embodiments.

FIG. 38 depicts a device in accordance with the present invention in use as applied to a pregnant user. In the depicted embodiment, the wings are first applied over the belly and attached to each other by Velcro®. The arms are applied and positioned at least partly over the wings and attached to one another, also by Velcro®.

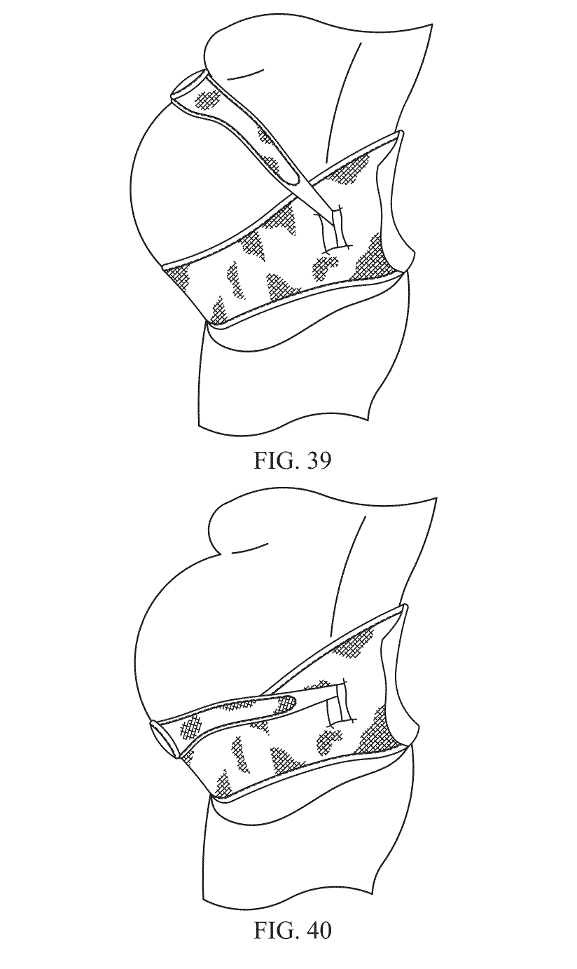

FIG. 39 depicts a device in accordance with the present invention in use as applied to a different pregnant user than in FIG. 38. In this embodiment, the wings are applied toward the bottom of the belly and attached to one another and the arms are positioned over the top of the belly and attached to each other in that position. The arms may be positioned over the belly, e.g., during late pregnancy. Positioning the device such that the arms rest upon the upper belly provides additional support and prevents the brace from slipping under the belly too far.

FIG. 40 depicts a device in accordance with the present invention in use as applied to a pregnant user. In the depicted embodiment, the wings are first applied over the belly and attached to one another, and the arms are applied and positioned at least somewhat over the wings and attached to one another.

According to non-limiting example embodiments, the support may be used for specified periods of time. According to non-limiting example embodiments, the present support devices may be used for example on a pregnant, human female throughout a woman's pregnancy, e.g., daily (e.g., 8 to 10 hours a day, 5 to 7 days a week), weekly (for example, several hours a day, one or two days a week), periodically (e.g., for four hours every morning or evening, or for increasing periods of time as the pregnancy progresses) or occasionally (for example during a portion of the day, when at work, when standing, when sitting for long periods of time, e.g., at the computer, on long car rides, when leaning over for greater than five minutes, when performing house chores, when gardening, when back pain starts to arise, or during other times of excess stress on one's back). In embodiments in which the present support or brace devices are used during a woman's pregnancy, the device may be adjusted to naturally contour/conform to the woman's anatomy, which changes throughout pregnancy.

The present lumbar support devices may optionally include electronics incorporated therein (e.g. incorporated in the fabric or between layers of fabric, which are capable of detecting and/or monitoring one or more maternal and/or fetal vital signs. Non-limiting examples of such vital signs may include, but are not limited to, fetal heart rate, maternal heart rate, fetal and maternal respiratory rate, placental blood flow and fetal/maternal EKG.

According to non-limiting example embodiments, electronics may be incorporated into the device that may include a timing and/or distance counter (e.g. pedometer) which may determine e.g., the length of time the brace has been worn on a particular day, and/or the distance or number of steps that a wearer has walked while wearing the device. An example timer may either count up or down, to e g simply indicate to a user how long the device has been worn (so as not to exceed recommended wear time), or to count down the amount of time worn.

The electronics may have a method of recording, displaying, and/or communicating such vital signs (or data). Data or calculated information (e.g. relating to vital signs, timing and/or distance) may be communicated to the wearer or other person (e.g. a nurse, doctor or family member) e.g. by viewing a display for example on the device, or viewing the information on e.g. personal electronic device, such as a smart phone, fitness tracker, or other device to which the information is communicated (e.g. using blue tooth or other wired or wireless communication method), or on a computer to which the data has been communicated. Other methods of communication are also known in the art, including for example vibrations, lights, sounds and other methods for communicating information. For example, if fetal or maternal vital signs are not within a preferred range, a sound may be made or a vibration made, or a message may be sent by email or text to the wearer or a medical professional.

Either raw or calculated data collected from the device may be provided to a medical professional, either directly (without additional affirmative action), or sent by the user (e.g. via email or internet connection—e.g. transferred by blue tooth in the doctor's office), so that software may process the information to ensure that vital signs are within a desired range and to potentially diagnose potential abnormalities or medical issues with the mother and/or baby.

Further provided are systems that include at least one lumbar support device, which includes electronics incorporated therein or thereon. The electronics may be for example, capable of detecting and/or monitoring one or more maternal and/or fetal vital signs as discussed above. The electronics may have a method of recording, displaying, and/or communicating such vital signs (or data). The systems may also include a smartphone or computer app or software that is capable of communicating with the lumbar support device either wired or wirelessly via a cord, wireless internet connection, Bluetooth or other method.

The adjustability of the present support devices maximizes the support's effectiveness because it is form fitted to the rapidly changing anatomy caused by pregnancy. Ultimately, this decreases the stresses on the spine, improving posture/alignment during activities of daily living.

According to example embodiments, the devices may be applied to a person either by the person themselves or with the assistance of another person. The devices may be applied by a method that includes positioning the device on the person such that a posterior panel(s) (when used) or middle portion of the wings is positioned across a back of the person. Such positioning may include for example, wings of the device overlapping the wings and attaching them to one another around the waist of the user. The wings may be attachable e.g., with buttons, zipper, hook and eye, snaps, hook and loop (e.g., Velcro®), or another fastener. If a front panel is present, the front panel may be adjusted, e.g. by moving the panel to a preferred position. Once positioned correctly, one may close the wings over the abdomen of the wearer.

The present methods may also include pulling the arms of the device from their positions through slits in the wings until they are at a desired length to be positioned around, over, or under the belly and then attaching the arms to one another.

By way of non-limiting example, the device may be applied to a wearer by a method comprising positioning the device on the person such that a posterior portion of a brace is across a back of the user; positioning and attaching wings over the abdomen of the person, and positioning and attaching arms having an elastomeric corset assembly, together across, over or under the belly to a desired position and tension.

In non-limiting example embodiments, it may be advantageous to close and position the arms before closing and positioning the wings.

According to non-limiting examples, when one or more panels are used, the plastic that forms the rigid side in an orthotics oven may be heated on for 15 minutes, such that it is malleable. Once the panel is heated and malleable, it may be positioned against the patient's back and held until the plastic is set to mold to the desired shape. One may want to push the back panel out to achieve lordosis to relieve tension.

According to non-limiting examples, when one or more panels are used, the side of the panels with the attachment mechanism (e.g. Velcro) should be attached to the brace. The large backpanel, an example of which is depicted in FIG. 46, can be directly attached to an inner midline portion of the brace (e.g. at fabric portion 10) of the brace. Front panels may optionally be attached to the brace device in one or both of two positions. It can be attached directly to an inner side of the main support straps. It can also be attached to an inner side of the adjustable arms/wings after the main straps are fastened. In this position, the front panel will be between the adjustable arms and the wings/straps.

Once appropriately adjusted, the support device will function to pull in the lumbar vertebrae, increasing lordosis and placing the center of gravity in a more natural position.

Example embodiments provided herein are further directed to kits that may include at least one lumbar support device provided herein and instructions for use of the device.

Such instructions may include for example instructions regarding proper positioning and application of the device on a person and/or methods of adjusting the device to achieve a proper fit. Example instructions may provide guidance with respect to when and how long to wear the device to achieve the best results. Further example instructions may provide instructions regarding visiting a physician when necessary and/or exercises the user may perform even when not wearing the device to improve muscle stability, help strengthen core and/or back muscles and help prevent and/or lessen back pain by methods in addition to wearing the device. Further example instructions may include instructions for cleaning the device.

Kits may also include devices according to present embodiments and one or more removable panels that may be attached to the device, as set forth herein.

Kits that include the present devices having electronics incorporated therein and/or thereon may also include software, software codes, a device or measuring or displaying or otherwise communicating data (such as the type of data discussed above), a cord for charging any electronics, and/or a device, such as a fitness tracker or smart phone for receiving data, alters or other communication.

The removable padded panel(s) may be selected from the group consisting of anterior, posterior and coronal panels.

In the present kits, the removable padded panels are preferably selected from panels that enable the brace to comply with L0627, L0631 and/or L0637 US brace codes, e.g. for post partum and non-pregnant individuals.

After the wings are positioned over a person's abdomen and attached to one another by a fastener, then the wearer may further secure or adjust the device by adjusting the arms. The arms may be secured and adjusted for example by pulling on the respective arms e.g., anteriorly (toward the front), which pulls on the arms and corset, and therefore, tighten the arms over the front of the body to help it conform to the belly and back.

Although the invention has been described in example embodiments, those skilled in the art will appreciate that various modifications may be made without departing from the spirit and scope of the invention. It is therefore to be understood that the inventions herein may be practiced other than as specifically described. Thus, the present embodiments should be considered in all respects as illustrative and not restrictive. Accordingly, it is intended that such modifications fall within the scope of the present invention as defined by the claims appended hereto.

What is claimed is:

1. A lumbar support device comprising
at least two wings having wing fasteners to enable the wings to fasten to each other across a belly of a user; and
at least two arms connected to each other by an elastic corset assembly, wherein the arms emerge from slits in the wings, said arms having arm fasteners to enable the arms to fasten to each other across, over, or under the belly of the user.

2. The lumbar support device of claim 1, wherein the wing fasteners are hook and loop fasteners.

3. The lumbar support device of claim 1, wherein the arm fasteners are hook and loop fasteners.

4. The lumber support device of claim 1, further comprising at least one panel fastener configured for attachment of at least one padded panel to the device.

5. The lumber support device of claim 4, wherein a panel fastener comprises a hook or loop portion.

6. The lumbar support device of claim 1, further comprising at least one removable padded panel selected from an anterior panel, a posterior panel and a coronal panel.

7. The lumbar support device of claim 6, wherein said at least one removable padded panel is attached to the device by at least one panel fastener.

8. The lumbar support device of claim 1, further comprising at least one panel configured with an opening to attach to the device of claim 1 by sliding the panel onto the device.

9. The lumbar support device of claim 1, wherein the lumbar support complies with L0627, L0631 and/or L0637 US brace codes.

10. The lumbar support device of claim 6, wherein the anterior panel is fastened to winged arms via a hook and loop fastener attachment between winged arms and the actual brace itself.

11. The lumbar support device of claim 6, wherein the anterior panel is configured to be placed under the support with hook and loop attachments to allow positioning by a user in a desired area for comfort.

12. The lumbar support device of claim 1, further comprising an electronic component capable of measuring, calculating and/or monitoring one or more forms of data selected from the group consisting of maternal vital signs, fetal vital signs, time, and distance.

13. A kit comprising
(1) a lumbar support device of claims 1; and
(2) instructions for using the lumbar support device.

14. The kit of claim 13, further comprising at least one removable padded panel selected from the group consisting of anterior, posterior and coronal panels.

15. The kit of claim 13, wherein the removable padded panels are selected from panels that enable the brace to comply with L0627, L0631 and/or L0637 US brace codes.

16. The kit of claim 13, wherein said instructions include instructions regarding proper application, positioning and adjustment of the device on a person.

17. A method of preventing or treating back pain in a person, comprising applying the lumbar support device of claim 1 to a person, and securing the device to the person by fastening the wings to one another and fastening the arms to one another.

18. The method of claim 17, wherein the device is configured such that the fastened arm position with respect to a person to whom the device is applied, is variable.

19. The method of claim 18, wherein the arms are positioned with respect to the person such that the arms are fastened to one another so as to rest over the fastened wings.

20. The method of claim 19, wherein the arms are positioned with respect to the person such that the arms are fastened to one another so as to rest on the upper abdominal belly.
